(19) United States
(12) Patent Application Publication
(10) Pub. No.: US 2024/0238380 A1
(43) Pub. Date: Jul. 18, 2024

(54) COMPOSITION FOR COMBINATION THERAPY COMPRISING GROWTH DIFFERENTIATION FACTOR-15 VARIANT AND GLUCAGON-LIKE PEPTIDE-1 RECEPTOR AGONIST

(71) Applicant: YUHAN CORPORATION, Seoul (KR)

(72) Inventors: Seyoung LIM, Gyeonggi-do (KR); Ji Eun YANG, Gyeonggi-do (KR); Sukyung KIM, Seoul (KR); Bo Ra SIM, Gyeonggi-do (KR); Yunji LEE, Gyeonggi-do (KR); Do Hoon KIM, Gyeonggi-do (KR); Mi Kyeong JU, Gyeonggi-do (KR); Hyun Ho CHOI, Gyeonggi-do (KR); Han Na HONG, Gyeonggi-do (KR); Junhwan KIM, Seoul (KR)

(21) Appl. No.: 18/562,322

(22) PCT Filed: May 20, 2022
(86) PCT No.: PCT/KR2022/007242

§ 371 Date: Nov. 19, 2023

(30) Foreign Application Priority Data
May 21, 2021 (KR) 10-2021-0065563

(51) Int. Cl. A61K 38/26; A61K 38/18; A61P 3/04; A61P 3/06; A61P 3/10

(52) U.S. Cl. CPC A61K 38/26; A61K 38/1841; A61P 3/04; A61P 3/06; A61P 3/10

(57) ABSTRACT

The present invention relates to a pharmaceutical composition for the prevention or treatment of diabetes, obesity, dyslipidemia, or metabolic syndrome by administering in combination with a GLP-1 (glucagon-like peptide-1) receptor agonist, comprising a GDF15 (growth differentiation factor-15) variant, a long-acting GDF15 fusion protein, or a long-acting GDF15 fusion protein dimer as an active ingredient.

### BACKGROUND OF THE INVENTION

### Field of the Invention

The present invention relates to a pharmaceutical composition for the prevention or treatment of diabetes, obesity, dyslipidemia, or metabolic syndrome by administering in combination with a GLP-1 (glucagon-like peptide-1) receptor agonist, comprising a GDF15 (growth differentiation factor-15) variant, a long-acting GDF15 fusion protein, or a long-acting GDF15 fusion protein dimer as an active ingredient.

### Description of the Related Art

GDF15, called MIC-1 (macrophage inhibitory cytokine-1), PBMP (placental bone morphogenetic protein), or NAG-1 (nonsteroidal anti-inflammatory drug-activated gene-1), is a protein that is a member of the TGF-β superfamily (transforming growth factor-beta superfamily).

Recently, it has been reported that GDF15 induces loss of body weight by inhibiting dietary intake through binding to RET (ret proto-oncogene) and GFRAL (GDNF family receptor alpha-like) specifically expressed in brain tissue (Tsai V. W. et al., PLOS One 2013; 8 (2): e55174; U.S. Pat. No. 8,192,735). Moreover, in several studies, administration of GDF15 to a variety of obese animal models demonstrated an excellent weight loss effect, and additionally, metabolic advantages such as lowered blood glucose levels, reduced lipid levels, improved insulin resistance, and the like were observed.

However, wild-type GDF15 has a short half-life in the body, so there is a problem in that the frequency of administration thereof is high when used medically. Accordingly, development of a long-acting formulation for increasing the half-life of GDF15 in the body is underway.

Meanwhile, glucagon-like peptide-1 (GLP-1), which is a GLP-1 receptor agonist, is an incretin hormone secreted by intestinal L cells in response to nutrient ingestion in the intestinal tract or blood glucose concentration, particularly a hormone that stimulates strong insulin secretion. It has the characteristic of enhancing insulin secretion depending on the glucose concentration, so it has a strong insulin secretion stimulation effect but does not cause hypoglycemia, which is desirable.

GLP-1 serves to decrease movement of the upper digestive tract and suppress appetite, and also enables proliferation of existing cells of the pancreas. Specifically, GLP-1 acts on the pancreas to increase insulin secretion and decrease glucagon secretion, thereby exhibiting a blood-glucose-lowering effect, and delays the passage of food through the stomach and suppresses appetite due to action on the brain, thereby controlling blood glucose in a complex way and aiding in weight loss. In addition, it is able to have a positive effect on insulin sensitivity by improving the function of islet beta cells (Zander M. et al., Lancet 2002; 359:824-830).

However, limitations are imposed on the use of active GLP-1, having a very short half-life of about 2 minutes, as a therapeutic agent. In order to overcome this limitation, efforts have been made to maintain the concentration of active GLP-1 by inhibiting DPP-4 (dipeptidylpeptidase-4), which is a hydrolase that inactivates GLP-1, and to search for a peptide that has a structure similar to that of GLP-1 and is not hydrolyzed by DPP-4. Currently, DPP-4 inhibitors have been developed and used as drugs corresponding to the former, and in the latter case, GLP-1 fragments or GLP-1 analogues have been developed and used.

The present inventors have made great efforts to improve the preventive or therapeutic effect on diabetes, obesity, dyslipidemia, or metabolic syndrome and thus ascertained that a composition for combination therapy including a GDF15 variant exhibiting improved activity by introducing a mutation at a certain position of GDF15 and a GLP-1 receptor agonist may show a weight loss effect and a blood lipid reduction effect, thus culminating in the present invention.

### CITATION LIST

(Patent Document 1) U.S. Pat. No. 8,192,735 (2009 Jan. 1)
(Non-Patent Document 1) Tsai V. W. et al., PLOS One 2013; 8 (2)
(Non-Patent Document 2) Zander M. et al., Lancet 2002; 359:824-830

### SUMMARY OF THE INVENTION

It is an object of the present invention to provide a pharmaceutical composition for the prevention or treatment of diabetes, obesity, dyslipidemia, or metabolic syndrome by administering in combination with a GLP-1 (glucagon-like peptide-1) receptor agonist, comprising a GDF15 (growth differentiation factor-15) variant, a long-acting GDF15 fusion protein, or a long-acting GDF15 fusion protein dimer as an active ingredient.

In order to accomplish the above object, the present invention provides a pharmaceutical composition for preventing or treating diabetes, obesity, dyslipidemia, or metabolic syndrome, which includes a GDF15 (growth differentiation factor-15) variant represented by Formula (I) below, a long-acting GDF15 fusion protein, or a long-acting GDF15 fusion protein dimer as an active ingredient and a GLP-1 (glucagon-like peptide-1) receptor agonist that is administered in combination therewith:

$$N - terminal extension domain - core domain \qquad Formula ( I )$$

in Formula (I),
the N-terminal extension domain is a polypeptide including any one amino acid sequence selected from among SEQ ID NOS: 3 to 5; and
the core domain is a polypeptide including the amino acid sequence of SEQ ID NO: 20, or a polypeptide in which any one selected from the group consisting of the 15th amino acid, 50th amino acid, 58th amino acid, 97th amino acid, and combinations thereof in the amino acid sequence of SEQ ID NO: 20 is substituted with another amino acid,
in which arginine (R), which is the 15th amino acid, may be substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),
asparagine (N), which is the 50^{th} amino acid, may be substituted with alanine, arginine, aspartic acid, cysteine, glutamic acid, glutamine, glycine, histidine, isoleucine, leucine, lysine, methionine, phenylalanine, proline, serine, threonine, tryptophan, tyrosine, or valine,
serine (S), which is the 58^{th} amino acid, may be substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or
aspartic acid (D), which is the 97^{th} amino acid, may be substituted with alanine, arginine, asparagine, cysteine, glutamic acid, glutamine, glycine, histidine, isoleucine, leucine, lysine, methionine, phenylalanine, proline, serine, threonine, tryptophan, tyrosine, or valine.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 shows the results of measurement of functional activity of long-acting GDF15 fusion proteins (dimer, FM9-1, FM9-2, FM9-3, FM9-4, FM9-5, and FM9-6) depending on the linker type and length;

FIG. 2 shows the results of measurement of functional activity of long-acting GDF15 fusion proteins (dimer, FM11-1, FM11-2, FM11-3, FM11-4, FM11-5 and FM11-6) depending on the linker type and length;

FIG. 3 shows the results of measurement of body weight change (%) in diet-induced obese mice (DIO mice) upon repeated administration of a long-acting GDF15 fusion protein (dimer, FM9-6) alone and in combination with semaglutide;

FIG. 4 shows the results of measurement of cumulative food intake in diet-induced obese mice (DIO mice) upon repeated administration of a long-acting GDF15 fusion protein (dimer, FM9-6) alone and in combination with semaglutide (****p<0.0001 vs. obese control (DIO vehicle) (Two-way ANOVA));

FIG. 5 shows the results of improvement of blood lipid profiles or measurement of the blood lipid profiles in diet-induced obese mice (DIO mice) upon repeated administration of a long-acting GDF15 fusion protein (dimer, FM9-6) alone and in combination with semaglutide (###p<0.001 vs. normal control (normal vehicle) (Student's t-test) and **p<0.01, ***p<0.001, and ****p<0.0001 vs. obese control (DIO vehicle) (One-way ANOVA));

FIG. 6 shows the results of measurement of body weight change (8) in ob/ob mice upon repeated administration of a long-acting GDF15 fusion protein (dimer, FM9-6) alone and in combination with semaglutide;

FIG. 7 shows the results of measurement of cumulative food intake in ob/ob mice upon repeated administration of a long-acting GDF15 fusion protein (dimer, FM9-6) alone and in combination with semaglutide (****p<0.0001 vs. obese control (ob/ob vehicle) (Two-way ANOVA));

FIG. 8 shows the results of improvement of blood lipid profiles or measurement of the blood lipid profiles in ob/ob mice upon repeated administration of a long-acting GDF15 fusion protein (dimer, FM9-6) alone and in combination with semaglutide (###p<0.001 vs. normal control (normal vehicle) (Student's t-test) and *p<0.05, **p<0.01, ***p<0.001, and ****p<0.0001 vs. obese control (ob/ob vehicle) (One-way ANOVA));

FIG. 9 shows the results of measurement of the blood glucose lowering effect and improvement of insulin resistance in ob/ob mice upon repeated administration of a long-acting GDF15 fusion protein (dimer, FM9-6) alone and in combination with semaglutide (###p<0.001 vs. normal control (normal vehicle) (Student's t-test) and *p<0.05, **p<0.01, and ****p<0.0001 vs. obese control (ob/ob vehicle) (One-way ANOVA));

FIG. 10 shows the results of measurement of body weight change (8) in ob/ob mice upon repeated administration of 1 nmol/kg of a long-acting GDF15 fusion protein (dimer, FM9-6) alone and in combination with a high dose of semaglutide;

FIG. 11 shows the results of measurement of cumulative food intake in ob/ob mice upon repeated administration of 1 nmol/kg of a long-acting GDF15 fusion protein (dimer, FM9-6) alone and in combination with a high dose of semaglutide (****p<0.0001 vs. obese control (ob/ob vehicle) (Two-way ANOVA));

FIG. 12 shows the results of measurement of changes in random blood glucose levels (mg/dL) in ob/ob mice upon repeated administration of 1 nmol/kg of a long-acting GDF15 fusion protein (dimer, FM9-6) alone and in combination with a high dose of semaglutide;

FIG. 13 shows the results of oral glucose tolerance test (OGTT) and improvement of insulin resistance in ob/ob mice upon repeated administration of 1 nmol/kg of a long-acting GDF15 fusion protein (dimer, FM9-6) alone and in combination with a high dose of semaglutide (*p<0.05, and ****p<0.0001 vs. obese control (ob/ob vehicle) (One-way ANOVA));

FIG. 14 shows the results of improvement of blood lipid profiles or measurement of the blood lipid profiles in ob/ob mice upon repeated administration of 1 nmol/kg of a long-acting GDF15 fusion protein (dimer, FM9-6) alone and in combination with a high dose of semaglutide (*p<0.05, **p<0.01, ***p<0.001, and ****p<0.0001 vs. obese control (ob/ob vehicle) (One-way ANOVA));

FIG. 15 shows the results of measurement of body weight change (%) in diet-induced obese mice (DIO mice) upon repeated administration of a long-acting GDF15 fusion protein (dimer, FM9-6) alone and in combination with semaglutide or tirzepatide;

FIG. 16 shows the results of measurement of cumulative food intake in diet-induced obese mice (DIO mice) upon repeated administration of a long-acting GDF15 fusion protein (dimer, FM9-6) alone and in combination with semaglutide or tirzepatide (*p<0.05, and ****p<0.0001 vs. obese control (DIO vehicle) (Two-way ANOVA));

FIG. 17 shows the results of improvement of blood lipid profiles or measurement of the blood lipid profiles in diet-induced obese mice (DIO mice) upon repeated administration of a long-acting GDF15 fusion protein (dimer, FM9-6) alone and in combination with semaglutide or tirzepatide (###p<0.001 vs. normal control (normal vehicle) (Student's t-test) and **p<0.01, and ***p<0.001 vs. obese control (DIO vehicle) (One-way ANOVA)); and

FIG. 18 shows the results of gastric emptying using acetaminophen in normal rats upon single administration of a long-acting GDF15 fusion protein (dimer, FM9-6) and semaglutide alone or in combination (*p<0.05, **p<0.01, and ***p<0.001 vs. normal control (normal vehicle) (One-way ANOVA)).

### DETAILED DESCRIPTION OF THE INVENTION

An aspect of the present invention pertains to a pharmaceutical composition for preventing or treating diabetes, obesity, dyslipidemia, or metabolic syndrome by administering in combination with a GLP-1 (glucagon-like peptide-1) receptor agonist, comprising a GDF15 (growth differentiation factor-15) variant, a long-acting GDF15 fusion protein, or a long-acting GDF15 fusion protein dimer as an active ingredient.

Hereinafter, a detailed description will be given of the present invention.

### GDF15 Variant

A GDF15 variant represented by Formula (I) below is provided.

$$N - terminal extension domain - core domain \qquad Formula ( I )$$

In Formula (I),
the N-terminal extension domain is a polypeptide including any one amino acid sequence selected from among SEQ ID NOS: 3 to 5; and
the core domain is a polypeptide including the amino acid sequence of SEQ ID NO: 20, or a polypeptide in which any one selected from the group consisting of the 15^{th} amino acid, 50^{th} amino acid, 58^{th} amino acid, 97^{th} amino acid, and combinations thereof in the amino acid sequence of SEQ ID NO: 20 is substituted with another amino acid,
in which arginine (R), which is the 15^{th} amino acid, may be substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),
asparagine (N), which is the 50^{th} amino acid, may be substituted with alanine, arginine (R), aspartic acid, cysteine, glutamic acid, glutamine, glycine, histidine, isoleucine, leucine, lysine, methionine, phenylalanine, proline, serine, threonine, tryptophan, tyrosine, or valine,
serine (S), which is the 58^{th} amino acid, may be substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or
aspartic acid (D), which is the 97^{th} amino acid, may be substituted with alanine, arginine, asparagine, cysteine, glutamic acid, glutamine, glycine, histidine, isoleucine, leucine, lysine, methionine, phenylalanine, proline, serine, threonine, tryptophan, tyrosine, or valine.

As used herein, the term “core domain” refers to a polypeptide having the amino acid sequence from the 7^{th} amino acid to the 112^{th} amino acid in the amino acid sequence of GDF15 of SEQ ID NO: 1, and is a polypeptide including the amino acid sequence of SEQ ID NO: 20 or a polypeptide in which any one selected from the group consisting of the 15^{th} amino acid, 50^{th} amino acid, 58^{th} amino acid, 97^{th} amino acid, and combinations thereof in the amino acid sequence of SEQ ID NO: 20 is substituted with another amino acid. The first core domain may include the amino acid sequence of SEQ ID NO: 2.

Specifically, the core domain may include any one mutation selected from the group consisting of the following mutations (1) to (6):
(1) arginine (R), which is the 15^{th} amino acid in the amino acid sequence of SEQ ID NO: 20, is substituted with asparagine (N);
(2) asparagine (N), which is the 50^{th} amino acid in the amino acid sequence of SEQ ID NO: 20, is substituted with leucine (L);
(3) serine (S), which is the 58^{th} amino acid in the amino acid sequence of SEQ ID NO: 20, is substituted with lysine (K), arginine (R), asparagine (N), aspartic acid (D), glutamic acid (E), cysteine (C), or leucine (L);
(4) aspartic acid (D), which is the 97^{th} amino acid in the amino acid sequence of SEQ ID NO: 20, is substituted with leucine (L);
(5) asparagine (N), which is the 50^{th} amino acid, and aspartic acid (D), which is the 97^{th} amino acid, in the amino acid sequence of SEQ ID NO: 20, are substituted with cysteine (C) or serine (S); and
(6) arginine (R), which is the 15^{th} amino acid in the amino acid sequence of SEQ ID NO: 20, is substituted with asparagine (N), and serine (S), which is the 58^{th} amino acid, is substituted with lysine (K) or arginine (R).

Here, the core domain may include any one amino acid sequence selected from among SEQ ID NOS: 6 to 19.

The N-terminal extension domain is a domain bound to the N-terminus of the core domain, and may be a polypeptide including any one amino acid sequence selected from among SEQ ID NOS: 3 to 5.

As used herein, the expression “ΔN2” may also be represented as “delta N2”, and means that the first and second amino acids in the amino acid sequence of human GDF15 set forth in SEQ ID NO: 1 are deleted. ΔN2 may be represented as “NGDH” when expressed as an N-terminal extension domain.

As used herein, the expression “ΔN3, WS insertion, G4N, D5S, H6T” may also be represented as “delta N3, WS insertion, G4N, D5S, H6T”, and means that the first to third amino acids in the amino acid sequence of human GDF15 set forth in SEQ ID NO: 1 are deleted, tryptophan and serine are inserted at the deleted positions, glycine, which is the fourth amino acid, is substituted with asparagine, aspartic acid, which is the fifth amino acid, is substituted with serine, and histidine, which is the sixth amino acid, is substituted with threonine, respectively. The ΔN3, WS insertion, G4N, D5S, and H6T may be represented as “WSNST” when expressed as an N-terminal extension domain.

As used herein, the expression “ΔN3, G4N, D5S, H6T” may also be represented as “delta N3, G4N, D5S, H6T”, and means that the first to third amino acids in the amino acid sequence of human GDF15 set forth in SEQ ID NO: 1 are deleted, glycine, which is the fourth amino acid, is substituted with asparagine, aspartic acid, which is the fifth amino is acid, substituted with serine, and histidine, which is the sixth amino acid, is substituted with threonine, respectively. The ΔN3, G4N, D5S, and H6T may be represented as “NST” when expressed as an N-terminal extension domain.

The GDF15 variant may include an N-terminal extension domain including the amino acid sequence set forth in SEQ ID NO: 3 and a core domain including any one amino acid sequence selected from among SEQ ID NOs: 6 to 20. In addition, the GDF15 variant may include an N-terminal extension domain including the amino acid sequence set forth in SEQ ID NO: 4 and a core domain including any one amino acid sequence selected from among SEQ ID NOs: 6 to 20. Moreover, the GDF15 variant may include an N-terminal extension domain including the amino acid sequence set forth in SEQ ID NO: 5 and a core domain including any one amino acid sequence selected from among SEQ ID NOs: 6 to 19.

Preferably, the GDF15 variant includes an N-terminal extension domain including the amino acid sequence set forth in SEQ ID NO: 3 and a core domain including the amino acid sequence set forth in SEQ ID NO: 8, 9, or 20. In addition, the GDF15 variant includes an N-terminal extension domain including the amino acid sequence set forth in SEQ ID NO: 4 and a core domain including the amino acid sequence set forth in SEQ ID NO: 8, 9, or 20. In addition, the GDF15 variant includes an N-terminal extension domain including the amino acid sequence set forth in SEQ ID NO: 5 and a core domain including any one amino acid sequence selected from among SEQ ID NOS: 6, 7, and 10 to 19. Here, the GDF15 variant may include any one amino acid sequence selected from among SEQ ID NOs: 21 to 39.

### Long-Acting GDF15 Fusion Protein

A long-acting GDF15 fusion protein is configured such that the GDF15 variant and a human IgG Fc or a variant thereof are bound to each other.

The human IgG Fc or the variant thereof may be Fc of IgG1, IgG2, IgG3, or IgG4, or a variant thereof. Specifically, the human IgG1 Fc or the variant thereof may be a human IgG1 Fc or a variant thereof, and the human IgG1 Fc may include the amino acid sequence set forth in SEQ ID NO: 41.

The human IgG Fc or the variant thereof may be a fragment of an Fc including a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may be a fragment of an Fc including a CH2 domain and a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may be a fragment of an Fc including a partial hinge region, a CH2 domain, and a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may have an amino acid sequence that is 90%, 92%, 95%, 96%, 97%, 98%, 99%, or 100% identical to SEQ ID NO: 41.

The IgG Fc or the variant thereof includes a first polypeptide including an IgG1 Fc sequence, the IgG1 Fc sequence including a CH3 sequence including at least one engineered protuberance, and a second polypeptide including an IgG1 Fc sequence, the IgG1 Fc sequence including a CH3 sequence including at least one engineered cavity, in which the first polypeptide may be heterodimerized with the second polypeptide via positioning of the protuberance of the first polypeptide into the cavity of the second polypeptide.

Specifically, the first polypeptide may include an engineered protuberance that enables binding of another IgG Fc polypeptide (e.g. a second polypeptide) including an engineered cavity. The second polypeptide may include an engineered cavity that enables binding of another IgG Fc polypeptide (e.g. a first polypeptide) including an engineered protuberance. In addition, the protuberance of the first polypeptide and the cavity of the second polypeptide may be engineered into a CH3 domain of IgG Fc. Here, the protuberance of the first polypeptide and the cavity of the second polypeptide are neither linked nor bound to the GDF15 variant.

The engineered protuberance may include at least one substitution in the amino acid sequence of human IgG1 Fc having the amino acid sequence set forth in SEQ ID NO: 41. Here, the numbering of amino acid positions is based on an EU numbering scheme. The substitution may be present at a position selected from the group consisting of amino acid residues 347, 366, and 394. For example, the substitution may be any one selected from the group consisting of Q347W/Y, T366W/Y, T394W/Y, and combinations thereof. In addition, the engineered cavity may include at least one substitution of the corresponding amino acid in the human IgG1 Fc sequence, and the substitution may be present at a position selected from the group consisting of amino acid residues 366, 368, 394, 405, and 407. For example, the substitution may be any one selected from the group consisting of T366S, L368A, T394S, F405T/V/A, Y407T/V/A, and combinations thereof.

Preferably, the protuberance includes a T366W/Y substitution, and the cavity includes any one substitution selected from the group consisting of T366S, L368A, Y407T/V/A, and combinations thereof. For example, the protuberance may include a T366W/Y substitution and the cavity may include a Y407T/V/A substitution. Moreover, the protuberance may include a T366Y substitution and the cavity may include a Y407T substitution. The protuberance may include a T366W substitution and the cavity may include a Y407A substitution. The protuberance may include a T394Y substitution and the cavity may include a Y407T substitution.

The first polypeptide may include any one amino acid sequence selected from among SEQ ID NOs: 42, 44, and 46, and the second polypeptide may include any one amino acid sequence selected from among SEQ ID NOs: 43, 45, and 47.

The protuberance is referred to as a “knob” and the cavity is referred to as a “hole”.

The first polypeptide is an Fc ‘knob’ including an engineered protuberance, and the second polypeptide is an Fc ‘hole’ including an engineered protuberance. The first polypeptide and the second polypeptide may be physically bound to each other through either or both of non-covalent interactions (e.g. hydrophobic effects such as hydrophobic interactions between knob and hole regions of Fc) and covalent bonds (e.g. disulfide bonds, such as 1 or 2 or more disulfide bonds between hinge regions of Fc in the first polypeptide and the second polypeptide).

As used herein, the term “dimer” refers to a protein complex including at least two polypeptides. Each of these polypeptides includes an N-terminus and a C-terminus. At least two polypeptides may be linked to each other through either or both of covalent and non-covalent interactions (e.g. electrostatic effects, n-effects, van der Waals forces, and hydrophobic effects). The two polypeptides may have the same amino acid sequence or different amino acid sequences, and a complex having two polypeptides that are the same as each other is referred to as a homodimer, and a complex having two polypeptides that are different from each other is referred to as a heterodimer.

The human IgG Fc or the variant thereof may be a heterodimer including the first polypeptide and the second polypeptide, and the heterodimer may be a heterodimer using A-1 (SEQ ID NO: 42) and A-2 (SEQ ID NO: 43), a heterodimer using B-1 (SEQ ID NO: 44) and B-2 (SEQ ID NO: 45), or a heterodimer using C-1 (SEQ ID NO: 46) and C-2 (SEQ ID NO: 47).

In addition, the IgG Fc or the variant thereof may include an additional mutation in order to improve the properties of the long-acting GDF15 fusion protein. Specifically, an additional mutation may be included in the heterodimer composed of the first polypeptide and the second polypeptide.

For example, the IgG Fc or the variant thereof may include a mutation(s) that abrogates (e.g. decreases or eliminates) IgG effector function. Specifically, the Fc partner sequence may include a mutation (s) that abrogates effector functions such as complement-dependent cytotoxicity (CDC), antibody-dependent cellular cytotoxicity (ADCC), and antibody-dependent cellular phagocytosis (ADCP). For example, an IgG Fc using A-1 and A-2 or a variant (heterodimer) thereof may include E233A and L235A mutations in order to remove the IgG1 effector function. A heterodimer using B-1 and B-2 including the N297A mutation may be used to remove N-linked glycan. A heterodimer using C-1 and C-2 may include L234A, L235A, and N297A mutations in order to remove the IgG1 effector function and N-linked glycan.

The GDF15 variant and the IgG Fc or the variant thereof may be bound through binding of the C-terminus of the first polypeptide or the C-terminus of the second polypeptide of the IgG Fc or the variant thereof to the N-terminus of the GDF15 variant. In addition, the GDF15 variant and the IgG Fc or the variant thereof may be bound through binding of the N-terminus of the first polypeptide or the N-terminus of the second polypeptide of the IgG Fc or the variant thereof to the C-terminus of the GDF15 variant. Preferably, the GDF15 variant and the IgG Fc or the variant thereof are bound through binding of the C-terminus of the first polypeptide of the IgG Fc or the variant thereof to the N-terminus of the GDF15 variant.

In addition, the GDF15 variant and the IgG Fc or the variant thereof may be bound via a linker. The linker may be a peptide that t includes glycine, serine, alanine, lysine and glutamic acid residues and is composed of 10 to 50 amino acid residues. The linker may include (G4S)ₙ, in which n may be an integer of 1 to 10 or an integer of 2 to 7. For example, n may be 2, 3, 4, 5, 6, or 7. In an embodiment of the present invention, a linker including (G4S)₅ in which n is an integer of 5 is used.

However, the present invention is not limited thereto, and as an example of a suitable linker other than (G4S)ₙ, the linker may include GS(G4S)ₙ, GS(EEEA)ₙ, (EEEA)ₙ, GS(EAAAK)ₙ, (EAAAK)ₙ, or GSGGSS(PT)ₙ, in which n may be an integer of 1 to 10. In an embodiment of the present invention, a linker including GS(EEEA)₆ in which n is an integer of 6 or a linker including GS(EAAAK)₅ in which n is an integer of 5 is used.

Specifically, the linker may be
(SEQ ID NO: 48)
GGGGSGGGGSGGGGSGGGGSGGGGS,
(SEQ ID NO: 92)
GSGGGGSGGGGSGGGGS,
(SEQ ID NO: 93)
GSGGGGSGGGGSGGGGSGGGGSGGGGS,
(SEQ ID NO: 94)
GSGGGGSGGGGSGGGGSGGGGSGGGGSGGGGSGGGGS,
(SEQ ID NO: 95)
GSEEEAEEEAEEEAEEEAEEEAEEEA,
(SEQ ID NO: 96)
GSGGSSPTPTPTPTPTPTPTPTPTPT,
or
(SEQ ID NO: 97)
GSEAAAKEAAAKEAAAKEAAAKEAAAK.
Preferably, the linker is
(SEQ ID NO: 48)
GGGGSGGGGSGGGGSGGGGSGGGGS,
(SEQ ID NO: 95)
GSEEEAEEEAEEEAEEEAEEEAEEEA,
or
(SEQ ID NO: 97)
GSEAAAKEAAAKEAAAKEAAAKEAAAK.

The long-acting GDF15 fusion protein includes one GDF15 variant per heterodimer composed of the first polypeptide and the second polypeptide. The GDF15 variant may include at least one N-linked glycan.

The long-acting GDF15 fusion protein may include i) a GDF15 variant including any one amino acid sequence selected from among SEQ ID NOs: 21 to 39, ii) a first polypeptide including any one amino acid sequence selected from among SEQ ID NOs: 42, 44, and 46, and iii) a second polypeptide including any one amino acid sequence selected from among SEQ ID NOs: 43, 45, and 47.

Preferably, the long-acting GDF15 fusion protein includes i) a GDF15 variant including any one amino acid sequence selected from among SEQ ID NOs: 21 to 39, ii) a linker including the amino acid sequence of SEQ ID NO: 48, iii) a first polypeptide including the amino acid sequence of SEQ ID NO: 42, and iv) a second polypeptide including the amino acid sequence of SEQ ID NO: 43.

More preferably, the long-acting GDF15 fusion protein includes i) a GDF15 variant including any one amino acid sequence selected from among SEQ ID NOs: 21 to 39, ii) a linker including any one amino acid sequence selected from among SEQ ID NOS: 92 to 97, iii) a first polypeptide including the amino acid sequence of SEQ ID NO: 46, and iv) a second polypeptide including the amino acid sequence of SEQ ID NO: 47.

### Fusion Protein Dimer

A long-acting GDF15 fusion protein dimer includes two long-acting GDF15 fusion proteins. Specifically, the two long-acting GDF15 fusion proteins are dimerized through GDF15-GDF15 interaction to form what is called a “fusion protein dimer”.

### Complex

The complex comprises a growth differentiation factor-15 (GDF15) variant and an IgG Fc, said complex being represented by the following formula (II):

$$IgG Fc - {( L )}_{m} - N - terminal extension domain - core domain \qquad ( II )$$

wherein m is an integer of 0 or 1,
L is a linker selected from the group consisting of SEQ ID NOs: 48, 92, 93, 94, 95, 96, and 97,
IgG Fc comprises the amino acid sequence of SEQ ID NOs: 42, 44, or 46, and
N-terminal extension domain and core domain are as defined in the above.

In an embodiment, the N-terminal extension domain-core domain comprises any one amino acid sequence selected from the group consisting of SEQ ID NOS: 21 to 39.

Further, the complex comprises any one amino acid sequence selected from the group consisting of SEQ ID NOS: 50-91 and 98-109.

### Nucleic Acid Molecule, Expression Vector, and Host Cell

The present invention pertains to an isolated nucleic acid molecule encoding the GDF15 variant or the long-acting GDF15 fusion protein.

As used herein, the term “isolated nucleic acid molecule” refers to a nucleic acid molecule of the present invention that has been separated from at least about 50% of proteins, lipids, carbohydrates, or other materials with which it is naturally found when total nucleic acid is isolated from the source cells, is operably linked to a polynucleotide to which it is not linked in nature, or does not occur in nature of as part a larger polynucleotide sequence. Specifically, the isolated nucleic acid molecule of the present invention is substantially free from any other contaminating nucleic acid molecules or other contaminants that are found in the natural environment thereof that would interfere with the use thereof for polypeptide production or for related treatment, diagnosis, prevention, or research.

As such, the isolated nucleic acid molecule encoding the GDF15 variant or the long-acting GDF15 fusion protein may have different sequences due to codon redundancy. In addition, the isolated nucleic acid molecule may be appropriately modified depending on the purpose, so long as it is able to produce the GDF15 variant or the long-acting GDF15 fusion protein, or nucleotides may be added at the N-terminus or C-terminus thereof.

The present invention pertains to an expression vector including the isolated nucleic acid molecule encoding the GDF15 variant or the long-acting GDF15 fusion protein.

As used herein, the term “expression vector” refers to a vector containing a nucleic acid sequence suitable for transformation of a host cell and directing or controlling the expression of an inserted heterologous nucleic acid sequence. Examples of the vector include, but are not limited to, linear nucleic acids, plasmids, phagemids, cosmids, RNA vectors, viral vectors, and analogues thereof. Examples of such viral vectors include, but are not limited to, retroviruses, adenoviruses, and adeno-associated viruses.

As used herein, the term “expression of a heterologous nucleic acid sequence” or “expression” of a protein of interest refers to transcription of an inserted DNA sequence, translation of an mRNA transcript, and production of a fusion protein product or antibody or antibody fragment.

A useful expression vector may be RcCMV (Invitrogen, Carlsbad) or a variant thereof. A useful expression vector may include a human cytomegalovirus (CMV) promoter to promote continuous transcription of a gene of interest in mammalian cells and a bovine growth hormone polyadenylation signal sequence to increase the steady-state level of RNA after transcription.

The present invention pertains to a host cell including the expression vector.

As used herein, the term “host cell” refers to prokaryotic and eukaryotic cells into which the recombinant expression vector may be introduced. As used herein, the terms “transformed” and “transfected” refer to the introduction of a nucleic acid (e.g. a vector) into a cell through many techniques known in the art.

The host cell may be transformed or transfected with the DNA sequence of the present invention, and may be used for expression and/or secretion of a protein of interest. The host cell that may be used in the present invention may include immortal hybridoma cells, NS/0 myeloma cells, 293 cells, Chinese hamster ovary cells (CHO cells), HeLa cells, CAP cells (cells derived from human amniotic fluid), or COS cells.

### GLP-1 Receptor Agonist

A GLP-1 receptor agonist may refer to a molecule that confers activity to a GLP-1 receptor. The GLP-1 receptor agonist may include, for example, GLP-1, a fragment thereof, or an analogue thereof.

The GLP-1 may include native GLP-1 or recombinant GLP-1. “Native” may refer to a polypeptide having the same amino acid sequence as a polypeptide found in nature. “Recombinant” may mean that a polypeptide having an amino acid having a specific sequence is encoded by a nucleic acid expressing GLP-1.

The fragment of GLP-1 may refer to a biologically active polypeptide obtained after cleavage of at least one amino acid from the N-terminus and/or C-terminus of the GLP-1 compound. The fragment of GLP-1 may include, for example, GLP-1(7-37), GLP-1(7-36), or GLP-1(9-36). The amino terminus of native GLP-1(7-37) is designated residue 7 and the carboxy terminus thereof is designated residue 37, and the nomenclature used to describe GLP-1 (7-37) OH may also be applied to the GLP-1 fragment. For example, GLP-1(9-36) refers to a GLP-1 fragment obtained by cleaving two amino acids from the N-terminus and cleaving one amino acid from the C-terminus. For example, GLP-1 (7-36) refers to a GLP-1 fragment obtained by cleaving one amino acid from the C-terminus.

The GLP-1 analogue may have a form in which at least one amino acid sequence of GLP-1 is modified to be different from the original sequence, an amino acid is cleaved therefrom, or an amino acid is added thereto.

Examples of the GLP-1 receptor agonist may include liraglutide (Novo Nordisk VICTOZA®); albiglutide (GlaxoSmithKline SYNCRIA®); taspoglutide (Hoffman La-Roche); dulaglutide (LY2189265); LY2428757; desamino-His⁷,Arg²⁶,Lys³⁴(N^{ε}-(γ-Glu(N-α-hexadecanoyl)))-GLP-1 (7-37); desamino-His⁷,Arg²⁶,Lys³⁴(N^{ε}-octanoyl)-GLP-1 (7-37); Arg^{26,34},Lys³⁸(N^{ε}-(ω-carboxypentadecanoyl))-GLP-1 (7-38); Arg^{26,34},Lys³⁶(N^{ε}-(γ-Glu(N-α-hexadecanoyl)))-GLP-1 (7-36); Aib^{8,35},Arg^{26,34},Phe³¹-GLP-1 (7-36); HXaa₈EGTFTSDVSSYLEXaa₂₂Xaa₂₃AAKEFIXaa₃₀WLXaa₃₃Xaa₃₄G Xaa₃₆Xaa₃₇ (in which Xaa₃ is A, V, or G; Xaa₂₂ is G, K, or E; Xaa₂₃ is Q or K; Xaa₃₀ is A or E; Xaa₃₃ is V or K; Xaa₃₄ is K, N, or R; Xaa₃₆ is R or G; Xaa₃₇ is G, H, P or none); Arg³⁴-GLP-1 (7-37); Glu³⁰-GLP-1(7-37); Lys²²-GLP-1(7-37); Gly^{8,36},Glu²²-GLP-1(7-37); Val⁸,Glu²²,Gly³⁶-GLP-1 (7-37); Gly^{8,36},Glu²²,Lys³³,Asn³⁴-GLP-1 (7-37); Val⁸,Glu²²,Lys³³,Asn³⁴,Gly³⁶-GLP-1 (7-37); Gly^{8,36},Glu²²,Pro³⁷-GLP-1(7-37); Val⁸,Glu²²,Gly³⁶Pro³⁷-GLP-1 (7-37); Gly^{8,36},Glu²²,Lys³³,Asn³⁴,Pro³⁷-GLP-1 (7-37); Val⁸,Glu²²,Lys³³,Asn³⁴,Gly³⁶,Pro³⁷-GLP-1 (7-37); Gly^{8,36},Glu²²-GLP-1(7-36); Val⁸,Glu²²,Gly³⁶-GLP-1 (7-36); Val⁸,Glu²²,Asn³⁴,Gly³⁶-GLP-1 (7-36); and Gly^{8,36},Glu²²,Asn³⁴-GLP-1(7-36). The specific sequence of the exemplified peptide is described in U.S. patent Ser. No. 10/905,772, which is incorporated herein by reference.

Examples of the GLP-1 receptor agonist may include exendin-4; exendin-3; Leu¹⁴-exendin-4; Leu¹⁴,Phe²⁵-exendin-4; Leu¹⁴,Ala¹⁹,Phe²⁵-exendin-4; exendin-4 (1-30); Leu¹⁴-exendin-4 (1-30); Leu¹⁴,Phe²⁵-exendin-4 (1-30); Leu¹⁴,Ala¹⁹,Phe²⁵-exendin-4 (1-30); exendin-4 (1-28); Leu¹⁴-Leu¹⁴,Phe²⁵-exendin-4 (1-28); exendin-4 (1-28); Leu¹⁴,Ala¹⁹,Phe²⁵-exendin-4 (1-28); Leu¹⁴,Lys^{17,20},Ala¹⁹,Glu²¹,Phe²⁵,Gln²⁸-exendin-4; Leu¹⁴, Lys 17, 20,Ala¹⁹,Glu²¹,Gln²⁸-exendin-4; octylGly¹⁴,Gln²⁸-exendin-4; Leu¹⁴,Gln²⁸,octylGly³⁴-exendin-4; Phe⁴,Leu¹⁴,Gln²⁸,Lys³³,Glu³⁴,Ile^{35,36},Ser³⁷-exendin-4 (1-37); Phe⁴,Leu¹⁴,Lys^{17,20},Ala¹⁹,Glu²¹,Gln²⁸-exendin-4; Val¹¹,Ile¹³,Leu¹⁴,Ala¹⁶,Lys²¹,Phe²⁵-exendin-4; exendin-4-Lys⁴⁰; lixisenatide (Sanofi-Aventis/Zealand Pharma); CJC-1134 (ConjuChem, Inc.); [N^{e}-(17-carboxyheptadecanoic acid)Lys²⁰]exendin-4-NH₂; [N^{e}-(17-carboxyhepta-decanoyl)Lys³²]exendin-4-NH₂; [desamino-His¹,N^{e}-(17-carboxyheptadecanoyl)Lys²⁰]exendin-4-NH₂; [Arg^{12,27},NLe¹⁴,N^{e}-(17-carboxy-heptadecanoyl)Lys³²]exendin-4-NH₂; [N^{e}-(19-carboxy-nonadecanoylamino)Lys²⁰]-exendin-4-NH₂; [N^{e}-(15-carboxypentadecanoylamino)Lys²⁰]-exendin-4-NH₂; [N^{e}-(13-carboxytridecanoylamino)Lys²⁰]exendin-4-NH₂; [N^{e}-(11-carboxy-undecanoyl-amino)Lys²⁰]exendin-4-NH₂; exendin-4-Lys⁴⁰ (e-MPA)-NH₂; exendin-4-Lys⁴⁰ (e-AEEA-AEEA-MPA)-NH₂; exendin-4-Lys⁴⁰ (e-AEEA-MPA)-NH₂; exendin-4-Lys⁴⁰ (e-MPA)-albumin; exendin-4-Lys⁴⁰ (e-AEEA-AEEA-MPA)-albumin; and exendin-4-Lys⁴⁰ (e-AEEA-MPA)-albumin. The specific sequence of the exemplified peptide is described in U.S. patent Ser. No. 10/905,772, which is incorporated herein by reference.

The GLP-1 receptor agonist may include, for example, a GLP-1-receptor-activating compound. The GLP-1-receptor-activating compound may be at least one selected from the group consisting of, for example, exenatide, liraglutide, lixisenatide, albiglutide, dulaglutide, semaglutide, tirzepatide, cotadutide, and taspoglutide.

| SEQ | | |
|---|---|---|
| ID | | |
| No. | Type | Sequence |
| 110 | Exenatide | HGEGTFTSDLSKQMEEEAVRLFIEWLKNGGPSSGAPPPS |
| 111 | Liraglutide | HAEGTFTSDVSSYLEGQAAKEEFIAWLVRGRG |
| | | (gamma-E-palmitoyl at E21) |
| 112 | Lixisenatide | HGEGTFTSDLSKQMEEEAVRLFIEWLKNGGPSSGAPPSKKKKKK |
| 113 | Taspoglutide | HXEGTFTSDVSSYLEGQAAKEFIAWLVKXR |
| | | (X: α-Aminoisobutyric acid) |
| 114 | Semaglutide | HXEGTFTSDVSSYLEGQAAKEFIAWLVRGRG |
| | | (X: α-Aminoisobutyric acid) |
| 115 | Dulaglutide | HGEGTFTSDVSSYLEEQAAKEFIAWLVKGGGGGGGSGGGGSGGGGSAES |
| | | KYGPPCPPCPAPEAAGGPSVFLFPPKPKDTLMISRTPEVTCVVVDVSQE |
| | | DPEVQFNWYVDGVEVHNAKTKPREEQFNSTYRVVSVLTVLHQDWLNGKE |
| | | YKCKVSNKGLPSSIEKTISKAKGQPREPQVYTLPPSQEEMTKNQVSLTC |
| | | LVKGFYPSDIAVEWESNGQPENNYKTTPPVLDSDGSFFLYSRLTVDKSR |
| | | WQEGNVFSCSVMHEALHNHYTQKSLSLSLG |
| 116 | Albiglutide | HGEGTFTSDVSSYLEGQAAKEFIAWLVKGRHGEGTFTSDVSSYLEGQAA |
| | | KEFIAWLVKGRDAHKSEVAHRFKDLGEENFKALVLIAFAQYLQQCPFED |
| | | HVKLVNEVTEFAKTCVADESAENCDKSLHTLFGDKLCTVATLRETYGEM |
| | | ADCCAKQEPERNECFLQHKDDNPNLPRLVRPEVDVMCTAFHDNEETFLK |
| | | KYLYEIARRHPYFYAPELLFFAKRYKAAFTECCQAADKAACLLPKLDEL |
| | | RDEGKASSAKQRLKCASLQKFGERAFKAWAVARLSQRFPKAEFAEVSKL |
| | | VTDLTKVHTECCHGDLLECADDRADLAKYICENQDSISSKLKECCEKPL |
| | | LEKSHCIAEVENDEMPADLPSLAADFVESKDVCKNYAEAKDVFLGMFLY |
| | | EYARRHPDYSVVLLLRLAKTYETTLEKCCAAADPHECYAKVFDEFKPLV |
| | | EEPQNLIKQNCELFEQLGEYKFQNALLVRYTKKVPQVSTPTLVEVSRNL |
| | | GKVGSKCCKHPEAKRMPCAEDYLSVVLNQLCVLHEKTPVSDRVTKCCTE |
| | | SLVNRRPCFSALEVDETYVPKEFNAETFTFHADICTLSEKERQIKKQTA |
| | | LVELVKHKPKATKEQLKAVMDDFAAFVEKCCKADDKETCFAEEGKKLVA |
| | | ASQAALGL |
| 117 | Tirzepatide | YXEGTFTSDYSIXLDKIAQKAFVQWLIAGGPSSGAPPPS |
| | | (X: α-Aminoisobutyric acid) |
| 118 | Cotadutide | HSQGTFTSDKSEYLDSERARDEVAWLEAGG |

In a specific embodiment of the present invention, when semaglutide or tirzepatide is used as a GLP-1-receptor-activating compound, an additive effect on weight loss and a significant decrease in triglyceride levels can be confirmed.

### Pharmaceutical Composition

The pharmaceutical composition according to the present invention may be administered through any route. The composition of the present invention may be provided to an animal either directly (e.g. by injection, transplantation, or local administration to a tissue site, topically) or systemically (e.g. parenterally or orally) through any suitable means. When the composition of the present invention is administered via an oral or parenteral route such as intravenous, subcutaneous, ophthalmic, intraperitoneal, intramuscular, intrarectal, intraorbital, intracerebral, intracranial, intraspinal, intraventricular, intrathecal, intracisternal, intracapsular, intranasal, or aerosol administration, the pharmaceutical composition may include, for example, an aqueous or physiologically applicable suspension of body fluids or a part of the solution thereof. Accordingly, the carrier or vehicle is physiologically acceptable and thus may be added to the composition and delivered to the patient. Therefore, it is generally possible to include physiological saline as a carrier for the formulation, like a body fluid.

The frequency of administration may also vary depending on the pharmacokinetic parameters of the GDF15 variant, long-acting GDF15 fusion protein, or long-acting GDF15 fusion protein dimer and the GLP-1 receptor agonist in the formulation that is used. Typically, the clinician will administer the pharmaceutical composition until a dosage that achieves the desired effect is reached. Thus, the pharmaceutical composition may be administered in a single dose or in two or more doses (which may or may not contain equivalent amounts of GDF15 variant, long-acting GDF15 fusion protein or long-acting GDF15 fusion protein dimer and GLP-1 receptor agonist) at temporal intervals, or through continuous infusion using a transplantation device or catheter. Additional refinement of the appropriate dosage may be routinely made by those skilled in the art, and falls within the realm of work routinely performed thereby.

Moreover, the unit dosage in humans is 0.01 μg/kg to 100 mg/kg, particularly 1 μg/kg to 10 mg/kg body weight. Although the above amount is optimal, it may vary depending on the disease to be treated and the presence or absence of side effects, and the optimal dosage may be determined through typical experimentation. Administration of the fusion protein may be based on periodic bolus injection or continuous intravenous, subcutaneous, or intraperitoneal administration from an external reservoir (e.g. an intravenous bag) or internal reservoir (e.g. a bioerodible implant).

### Administration in Combination

The GDF15 variant, the long-acting GDF15 fusion protein, or the long-acting GDF15 fusion protein dimer and the GLP-1 receptor agonist preferably have complementary activities, so they do not adversely affect each other.

The composition according to the present invention may (1) administered or delivered simultaneously through co-formulation in the form of a complex formulation or (2) may be administered or delivered simultaneously or sequentially as separate formulations.

In the complex formulation, the GDF15 variant, the long-acting GDF15 fusion protein, or the long-acting GDF15 fusion protein dimer and the GLP-1 receptor agonist may be present in the same composition. The formulation may be, for example, a dried powder composition, solution, or suspension, but is not limited thereto.

The GDF15 variant, the long-acting GDF15 fusion protein, or the long-acting GDF15 fusion protein dimer and the GLP-1 receptor agonist be administered may simultaneously or sequentially. The GDF15 variant, the long-acting GDF15 fusion protein, or the long-acting GDF15 fusion protein dimer and the GLP-1 receptor agonist are generally separated from each other, and may be administered simultaneously or sequentially. When administered sequentially, administration may be performed two or more times. When administered sequentially, the GDF15 variant, the long-acting GDF15 fusion protein, or the long-acting GDF15 fusion protein dimer and the GLP-1 receptor agonist may be administered one by one, or alternatively, the GDF15 variant, the long-acting GDF15 fusion protein, or the long-acting GDF15 fusion protein dimer and the GLP-1 receptor agonist may be administered two by two at temporal intervals.

A suitable dosage for the agonist may be an amount commonly used in the industry at present, and the dosage may be decreased due to the use of the GDF15 variant.

### Administration of Composition

The composition of the present invention may be administered through any route suitable for the disease to be treated. Suitable routes include oral, parenteral (including subcutaneous, intramuscular, intravenous, intraarterial, inhalation, intradermal, intrathecal, epidural, and infusion techniques), transdermal, intrarectal, intranasal, topical (including buccal and sublingual), vaginal, intraperitoneal, intrapulmonary, and intranasal administration. Topical administration may include the use of transdermal administration, such as a transdermal patch or iontophoresis device. The formulation of drugs is disclosed in Remington's Pharmaceutical Sciences, 18^{th} Ed., (1995) Mack Publishing Co., Easton, PA. Other examples of drug formulations are described in Liberman, H. A. and Lachman, L., Eds., Pharmaceutical Dosage Forms, Marcel Decker, Vol. 3, 2^{nd} Ed., New York, NY.

The preferred routes vary depending on, for example, the condition of the recipient. For oral administration, the composition may be formulated into a pill, capsule, tablet, etc. along with a pharmaceutically acceptable carrier, lubricant, or excipient. For parenteral administration, the composition may be formulated in the form of a unit dose injection along with a pharmaceutically acceptable parenteral vehicle or diluent.

### Prevention or Treatment

The composition of the present invention is used for the prevention or treatment of diabetes, obesity, dyslipidemia, or metabolic syndrome. The composition of the present invention may be used for the prevention or treatment of diseases, for example, type 1 diabetes, type 2 diabetes, diabetic neuropathy, diabetic nephropathy, diabetic retinopathy, diabetic cardiomyopathy, elevated elevated insulin levels, obesity, glucose levels, aggravated disease due to obesity, dyslipidemia, or metabolic syndrome (syndrome X or insulin resistance syndrome).

The present invention pertains to the use of the GDF15 variant, the long-acting GDF15 fusion protein, or the long-acting GDF15 fusion protein dimer and the GLP-1 receptor agonist for the prevention or treatment of diabetes, obesity, dyslipidemia, or metabolic syndrome. The present invention pertains to the use of the GDF15 variant, the long-acting GDF15 fusion protein, or the long-acting GDF15 fusion protein dimer and the GLP-1 receptor agonist for the manufacture of a medicament for the prevention or treatment of diabetes, obesity, dyslipidemia, or metabolic syndrome. The present invention pertains to a method of preventing or treating diabetes, obesity, dyslipidemia, or metabolic syndrome including administering to a subject the GDF15 variant, the long-acting GDF15 fusion protein, or the long-acting GDF15 fusion protein dimer and the GLP-1 receptor agonist.

The subject may be a subject suffering from diabetes, obesity, dyslipidemia, or metabolic syndrome. Moreover, the subject may be a mammal, preferably a human.

The term “treatment” refers to any indication of success in the treatment or amelioration of an injury, pathology, or condition, including any subjective or objective parameter such as abatement, remission, diminishing of symptoms, injury, pathology or condition more tolerable to the patient, slowing of the rate of regression or decline, creation of a final point of regression that is less debilitating, and improvement of a patient's physical or mental well-being. The treatment or amelioration of symptoms may be based on any objective or subjective parameter including physical examination, neuropsychiatric examination, and/or psychiatric evaluation.

The “effective amount” is an amount generally sufficient to reduce the severity or frequency of symptoms, eliminate the symptom or underlying cause, prevent the occurrence or underlying cause of the symptom, or ameliorate or correct any damage resulting from or associated with a disease state. In some embodiments, the effective amount is a therapeutically effective amount or a prophylactically effective amount. The “therapeutically effective amount” is an amount sufficient to correct a disease state or symptom, particularly a condition or symptom associated with the disease state, or to prevent, impede, delay, or reverse the progression of a disease state or any other undesirable symptom associated in any way with the disease. The “prophylactically effective amount” is the amount of a pharmaceutical composition that, when administered to a subject, has an intended prophylactic effect, such as preventing or delaying the onset of a disease state, or reducing the likelihood of onset (or recurrence) of a disease state or related symptoms. The therapeutically effective amount of the composition according to the present invention may be the amount of the GDF15 variant, the long-acting GDF15 fusion protein, or the long-acting GDF15 fusion protein dimer and the GLP-1 receptor agonist supporting reduction in an observable level of a biological or medical response, such as blood glucose, insulin, triglyceride, or cholesterol levels, weight loss, or improvements in glucose tolerance, energy expenditure, or insulin sensitivity.

### Product

A kit including the GDF15 variant, the long-acting GDF15 fusion protein, or the long-acting GDF15 fusion protein dimer and the GLP-1 receptor agonist useful for the treatment of the diseases and disorders described above may be provided. The kit includes a single container upon co-formulation in a complex formulation form. The kit includes a container containing each of the GDF15 variant, the long-acting GDF15 fusion protein, or the long-acting GDF15 fusion protein dimer and the GLP-1 receptor agonist.

The kit may further include a label or package insert provided with or affixed to the container. The term “package insert” may refer to instructions commonly included within commercial packages for therapeutic products, and contains information regarding the indications, use, dosage, administration, contraindications and/or warnings regarding the use of the therapeutic product. Suitable containers include, for example, bottles, vials, syringes, blister packs, and the like. The container may be formed from a variety of materials, such as glass or plastic. The label or package insert indicates that the composition is used for the prevention or treatment of a particular disease, such as diabetes, obesity, dyslipidemia, or metabolic syndrome. Additionally, it may further include a second container including a pharmaceutically acceptable buffer, such as bacteriostatic water for injection (BWFI), phosphate-buffered saline, Ringer's solution, and a dextrose solution. It may further include other materials desirable from a commercial and user standpoint, such as other buffers, diluents, fillers, needles, and syringes.

The kit may further include instructions for administering the GDF15 variant, the long-acting GDF15 fusion protein, or the long-acting GDF15 fusion protein dimer and GLP-1 receptor agonist simultaneously, sequentially, or separately to a patient in need thereof.

A better understanding of the present invention may be obtained through the following examples. However, these examples may be modified into various other forms, and are not to be construed as limiting the scope of the present invention.

### Example 1: Production of Long-Acting GDF15 Fusion Protein

### Example 1.1: Gene Cloning

For fusion carrier and linker optimization studies on two variants (FM9+Fc_hole, FM11+Fc_hole) showing excellent functional activity and improved purity after purification, long-acting GDF15 fusion proteins in which fusion carriers with minimized effector functions (SEQ ID NOs: 46 and 47) and various linkers (SEQ ID NOS: 92, 93, 94, 95, 96, and 97) were designed, and are shown in Table 1 below.

TABLE 1

| Material | | | |
|---|---|---|---|
| representation | | | |
| (Sequence | GDF15 | | |
| number) | Sequence change | Fusion carrier | Linker sequence |
| FM9-1 + Fc_hole | ΔN2, S64R | IgG1 Fc_knob (SEQ ID NO: 46) | GS(G4S)₃ |
| (SEQ ID NOs: 98 | (SEQ ID NO: 28) | IgG1 Fc_hole (SEQ ID NO: 47) | (SEQ ID NO: 92) |
| and 47) | | | |
| FM9-2 + Fc_hole | ΔN2, S64R | IgG1 Fc_knob (SEQ ID NO: 46) | GS(G4S)₅ |
| (SEQ ID NOs: 99 | (SEQ ID NO: 28) | IgG1 Fc_hole (SEQ ID NO: 47) | (SEQ ID NO: 93) |
| and 47) | | | |
| FM9-3 + Fc_hole | ΔN2, S64R | IgG1 Fc_knob (SEQ ID NO: 46) | GS(G4S)₇ |
| (SEQ ID NOs: 100 | (SEQ ID NO: 28) | IgG1 Fc_hole (SEQ ID NO: 47) | (SEQ D NO: 94) |
| and 47) | | | |
| FM9-4 + Fc_hole | ΔN2, S64R | IgG1 Fc_knob (SEQ ID NO: 46) | GS(EEEA)₆ |
| (SEQ ID NOs: 101 | (SEQ ID NO: 28) | IgG1 Fc_hole (SEQ ID NO: 47) | (SEQ ID NO: 95) |
| and 47) | | | |
| FM9-5 + Fc_hole | ΔN2, S64R | IgG1 Fc_knob (SEQ ID NO: 46) | GSGGSS(PT)₁₀ |
| (SEQ ID NOs: 102 | (SEQ ID NO: 28) | IgG1 Fc_hole (SEQ ID NO: 47) | (SEQ ID NO: 96) |
| and 47) | | | |
| FM9-6 + Fc_hole | ΔN2, S64R | IgG1 Fc_knob (SEQ ID NO: 46) | GS(EAAAK)₅ |
| (SEQ ID NOs: 103 | (SEQ ID NO: 28) | IgG1 Fc_hole (SEQ ID NO: 47) | (SEQ ID NO: 97) |
| and 47) | | | |
| FM11-1 + Fc_hole | ΔN3, G4N, D5S, | IgG1 Fc_knob (SEQ ID NO: 46) | GS(G4S)₃ |
| (SEQ ID NOs: 104 | H6T, S64R | IgG1 Fc_hole (SEQ ID NO: 47) | (SEQ ID NO: 92) |
| and 47) | (SEQ ID NO: 30) | | |
| FM11-2 + Fc_hole | ΔN3, G4N, D5S, | IgG1 Fc_knob (SEQ ID NO: 46) | GS(G4S)₅ |
| (SEQ ID NOs: 105 | H6T, S64R | IgG1 Fc_hole (SEQ ID NO: 47) | (SEQ ID NO: 93) |
| and 47) | (SEQ ID NO: 30) | | |
| FM11-3 + Fc_hole | ΔN3, G4N, D5S, | IgG1 Fc_knob (SEQ ID NO: 46) | GS(G4S)₇ |
| (SEQ ID NOs: 106 | H6T, S64R | IgG1 Fc_hole (SEQ ID NO: 47) | (SEQ ID NO: 94) |
| and 47) | (SEQ ID NO: 30) | | |
| FM11-4 + Fc_hole | ΔN3, G4N, D5S, | IgG1 Fc_knob (SEQ ID NO: 46) | GS(EEEA)₆ |
| (SEQ ID NOs: 107 | H6T, S64R | IgG1 Fc_hole (SEQ ID NO: 47) | (SEQ ID NO: 95) |
| and 47) | (SEQ ID NO: 30) | | |
| FM11-5 + Fc_hole | ΔN3, G4N, D5S, | IgG1 Fc_knob (SEQ ID NO: 46) | GSGGSS(PT)₁₀ |
| (SEQ ID NOs: 108 | H6T, S64R | IgG1 Fc_hole (SEQ ID NO: 47) | (SEQ ID NO: 96) |
| and 47) | (SEQ ID NO: 30) | | |
| FM11-6 + Fc_hole | ΔN3, G4N, D5S, | IgG1 Fc_knob (SEQ ID NO: 46) | GS(EAAAK)₅ |
| (SEQ ID NOs: 109 | H6T, S64R | IgG1 Fc_hole (SEQ ID NO: 47) | (SEQ ID NO: 97) |
| and 47) | (SEQ ID NO: 30) | | |

Specifically, in order to produce a first polypeptide (FM series) having the structure of an Fc_knob-(G4S)₅-GDF15 variant a second polypeptide having an Fc_hole structure, gene cloning was performed using a pcDNA3.3 (Invitrogen) expression vector including a gene encoding a first polypeptide including any one amino acid sequence selected from among SEQ ID NOs: 98 to 109 and a gene encoding a second polypeptide including the amino acid sequence of SEQ ID NO: 47. Here, synthesis of the nucleotide sequences encoding the amino acid sequences of SEQ ID NOS: 98 to 109 and SEQ ID NO: 47 was outsourced to Macrogen.

### Example 1.2: Expression and Purification of Long-Acting GDF15 Fusion Protein (Dimer)

On the 8^{th} day after transient transfection of an ExpiCHO cell line (Invitrogen) with the pcDNA3.3 expression vector cloned in Example 1.1, the cell culture fluid was harvested and purified. In order to purify the first and second polypeptides in the harvested cell culture fluid (HCCF), affinity purification was performed using Protein A resin.

Specifically, the harvested cell culture fluid was loaded on a MabSelect SuRe Protein A resin (GE Healthcare) equilibrated with 1×PBS (pH 7.4) to thus induce binding. After completion of binding of the first polypeptide and the second polypeptide, the MabSelect SuRe Protein A resin was washed with 1×PBS (pH 7.4), followed by elution using a 0.1 M glycine (pH 3.5) solution to obtain a final material.

The first polypeptide and the second polypeptide were neutralized to a pH of about 8.0 using a 1 M Tris-HCl solution. The first and second polypeptides were fully dimerized through a knock-in-hole interaction, and the result was named a “long-acting GDF15 fusion protein”. Two long-acting GDF15 fusion protein molecules were dimerized again through GDF15-GDF15 interaction, and the result was named a “fusion protein dimer”.

Also, in order to obtain a highly pure long-acting GDF15 fusion protein, a pool obtained after completion of first-step purification was subjected to second-step ion exchange (IEX) purification using an anion exchange (AEX) resin and a cation exchange (CEX) resin.

Specifically, for anion exchange (AEX), the pool obtained after the first step above was loaded on a POROS HQ 50 μm Strong Anion Exchange resin (Thermo Fisher) equilibrated with 1×PBS (pH 7.4) to thus induce binding. After completion of binding of the first polypeptide and the second polypeptide, the POROS HQ 50 μm Strong Anion Exchange resin was washed with 1×PBS (pH7.4), followed by concentration gradient elution using a 50 mM Tris-HCl (pH 8.0) solution containing 1 M sodium chloride to obtain a final material. Fractions having purity of 95% or higher were pooled using size-exclusion chromatography analysis.

In addition, for cation exchange (CEX), the pool obtained after the first step above was subjected to pH adjustment depending on the isoelectric point and then loaded on a POROS XS Strong Cation Exchange resin (Thermo Fisher) equilibrated with a 20 mM sodium phosphate (pH 6.5) solution to thus induce binding. After completion of binding of the first polypeptide and the second polypeptide, the POROS XS Strong Cation Exchange resin was washed with a 20 mM sodium phosphate (pH 6.5) solution, followed by concentration gradient elution using a 20 mM sodium phosphate (pH 6.5) solution containing 1 M sodium chloride to obtain a final material. Fractions having purity of 95% or more were pooled using size-exclusion chromatography analysis.

### Example 2: Measurement of Functional Activity of Long-Acting GDF15 Fusion Protein (Dimer)

Two variants (FM9+Fc_hole, FM11+Fc_hole) were compared and evaluated for GDF15 activity depending on the linker type and length. The GDF15 activity was measured using a Bright-Glo™ luciferase assay kit (Promega) and a GFRAL/RET/SRE-luc-overexpressing HEK293 cell line (Human embryonic kidney 293).

Specifically, 1×10⁵ GFRAL/RET/SRE-luc-overexpressing HEK293 cells were dispended in DMEM containing 10% FBS in each well of a 96-well-plate, followed by culture at 37° C. and 5% CO₂ for 24 hours. After 24 hours, each medium of the 96-well plate was replaced with 50 μl of a serum-free medium, followed by culture at 37° C. and 5% CO₂ for 4 hours.

In addition, the long-acting GDF15 fusion protein produced in Example 1 was prepared by 3-fold serial dilution from a concentration of 2000 nM using a serum-free medium. Thereafter, 50 μl of the long-acting GDF15 fusion protein dilution was added to each well containing 50 μl of the replaced serum-free medium and the GERAL/RET/SRE-luc cell line so that the actual concentration thereof was serially diluted 3-fold from 1000 nM, followed by reaction at 37° C. and 5% CO₂ for 4 hours. After 4 hours, each well was treated with 100 μl of a Bright-Glo™ solution prepared by adding a Bright-Glo™ buffer to a Bright-Glo™ substrate, followed by reaction at room temperature for 1 minute.

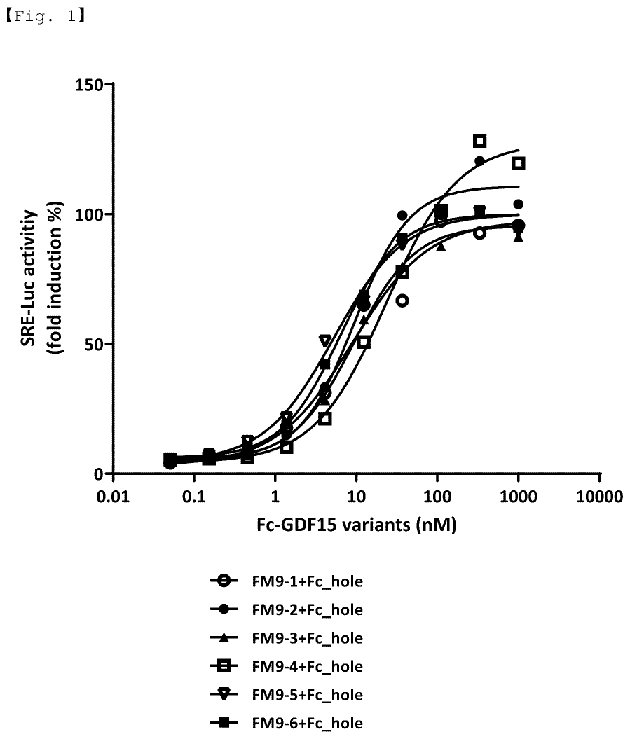

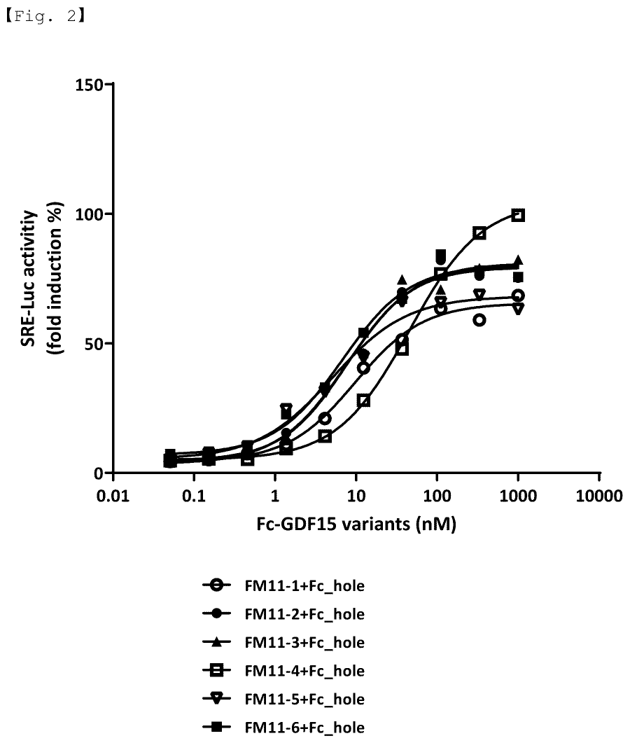

Thereafter, the reaction value (relative light units, RLU) was measured using a microplate reader (Perkin Elmer, Wallac Victor X5) capable of measuring luminescence. The results thereof are shown in Table 2 below and in FIGS. 1 and 2. Here, the functional activity of the long-acting GDF15 fusion protein depending on the GDF15 sequence, linker type, and length was compared based on the in-vitro GDF15 activity of FM9-6+Fc_hole (Eₘₐₓ 100%).

TABLE 2

| Material | GDF15 | | | | |
|---|---|---|---|---|---|
| representation | Sequence | | Linker | Emax | EC50 |
| (Sequence number) | change | Fusion carrier | sequence | (%) | (nM) |
| FM9-1 + Fc_hole | ΔN2, S64R | IgG1 Fc_knob | GS(G4S)₃ | 97.6 | 9.0 |
| (SEQ ID NOS: 98 and | (SEQ ID NO: | (SEQ ID NO: 46) | (SEQ ID NO: 92) | | |
| 47) | 28) | IgG1 Fc_hole | | | |
| | | (SEQ ID NO: 47) | | | |
| FM9-2 + Fc_hole | ΔN2, S64R | IgG1 Fc_knob | GS(G4S)₅ | 110.8 | 9.5 |
| (SEQ ID NOS: 99 and | (SEQ ID NO: | (SEQ ID NO: 46) | (SEQ ID NO: 93) | | |
| 47) | 28) | IgG1 Fc_hole | | | |
| | | (SEQ ID NO: 47) | | | |
| FM9-3 + Fc_hole | ΔN2, S64R | IgG1 Fc_knob | GS(G4S)₇ | 95.6 | 9.3 |
| (SEQ ID NOs: 100 | (SEQ ID NO: | (SEQ ID NO: 46) | (SEQ ID NO: 94) | | |
| and 47) | 28) | IgG1 Fc_hole | | | |
| | | (SEQ ID NO: 47) | | | |
| FM9-4 + Fc_hole | ΔN2, S64R | IgG1 Fc_knob | GS(EEEA)₆ | 127.6 | 23.6 |
| (SEQ ID NOs: 101 | (SEQ ID NO: | (SEQ ID NO: 46) | (SEQ ID NO: 95) | | |
| and 47) | 28) | IgG1 Fc_hole | | | |
| | | (SEQ ID NO: 47) | | | |
| FM9-5 + Fc_hole | ΔN2, S64R | IgG1 Fc_knob | GSGGSS(PT)₁₀ | 100.0 | 5.3 |
| (SEQ ID NOs: 102 | (SEQ ID NO: | (SEQ ID NO: 46) | (SEQ ID NO: 96) | | |
| and 47) | 28) | IgG1 Fc_hole | | | |
| | | (SEQ ID NO: 47) | | | |
| FM9-6 + Fc_hole | ΔN2, S64R | IgG1 Fc_knob | GS(EAAAK)₅ | 100.0 | 6.3 |
| (SEQ ID NOs: 103 | (SEQ ID NO: | (SEQ ID NO: 46) | (SEQ ID NO: 97) | | |
| and 47) | 28) | IgG1 Fc_hole | | | |
| | | (SEQ ID NO: 47) | | | |
| FM11-1 + Fc_hole | ΔN3, G4N, | IgG1 Fc_knob | GS(G4S)₃ | 65.6 | 9.7 |
| (SEQ ID NOs: 104 | D5S, H6T, | (SEQ ID NO: 46) | (SEQ ID NO: 92) | | |
| and 47) | S64R (SEQ ID | IgG1 Fc_hole | | | |
| | NO: 30) | (SEQ ID NO: 47) | | | |
| FM11-2 + Fc_hole | ΔN3, G4N, | IgG1 Fc_knob | GS(G4S)₅ | 79.4 | 7.7 |
| (SEQ ID NOs: 105 | D5S, H6T, | (SEQ ID NO: 46) | (SEQ ID NO: 93) | | |
| and 47) | S64R (SEQ ID | IgG1 Fc_hole | | | |
| | NO: 30) | (SEQ ID NO: 47) | | | |
| FM11-3 + Fc_hole | ΔN3, G4N, | IgG1 Fc_knob | GS(G4S)₇ | 81.0 | 7.8 |
| (SEQ ID NOs: 106 | D5S, H6T, | (SEQ ID NO: 46) | (SEQ ID NO: 94) | | |
| and 47) | S64R (SEQ ID | IgG1 Fc_hole | | | |
| | NO: 30) | (SEQ ID NO: 47) | | | |
| FM11-4 + Fc_hole | ΔN3, G4N, | IgG1 Fc_knob | GS(EEEA)₆ | 104.8 | 45.4 |
| (SEQ ID NOs: 107 | D5S, H6T, | (SEQ ID NO: 46) | (SEQ ID NO: 95) | | |
| and 47) | S64R (SEQ ID | IgG1 Fc_hole | | | |
| | NO: 30) | (SEQ ID NO: 47) | | | |
| FM11-5 + Fc_hole | ΔN3, G4N, | IgG1 Fc_knob | GSGGSS(PT)₁₀ | 68.2 | 5.1 |
| (SEQ ID NOs: 108 | D5S, H6T, | (SEQ ID NO: 46) | (SEQ ID NO: 96) | | |
| and 47) | S64R (SEQ ID | IgG1 Fc_hole | | | |
| | NO: 30) | (SEQ ID NO: 47) | | | |
| FM11-6 + Fc_hole | ΔN3, G4N, | IgG1 Fc_knob | GS(EAAAK)₅ | 80.3 | 6.8 |
| (SEQ ID NOs: 109 | D5S, H6T, | (SEQ ID NO: 46) | (SEQ ID NO: 97) | | |
| and 47) | S64R (SEQ ID | IgG1 Fc_hole | | | |
| | NO: 30) | (SEQ ID NO: 47) | | | |

As is apparent from the results of Table 2, each long-acting GDF15 fusion protein showed similar activity except for the linker GS(EEEA)₆ (SEQ ID NO: 95), and the long-acting GDF15 fusion proteins (FM9-4+Fc_hole, FM11-4+Fc_hole) linked via the linker GS(EEEA) 6 (SEQ ID NO: 95) were confirmed to show relatively low EC₅₀ values and high Eₘₐₓ values (FIGS. 1 and 2).

### Example 3: Evaluation of Pharmacokinetics of Long-Acting GDF15 Fusion Protein (Dimer)

6-week-old male C57BL/6N mice (Orient Bio, Republic of Korea) were purchased from Hallym Lab. Animal Inc., Republic of Korea. Animals were acclimated for 7 days, and on the day of drug administration, the mice were grouped based on the average body weight of individual mice (n=3 per blood sampling time per group). Then, FM9-4+Fc_hole, FM9-6+Fc_hole, FM11-4+Fc_hole, and FM11-6+Fc_hole were subcutaneously administered at 1 mg/kg to each group, and blood samples were collected at 4, 24, 48, 72, 96, 120, 168, and 240 hours after administration. Determination of active GDF15 in serum was achieved by enzyme-linked immunosorbent assay (ELISA), and pharmacokinetic parameters of each long-acting GDF15 fusion protein (dimer) are shown in Table 3 below.

TABLE 3

| Material | | | CL/F | Vd/F | AUCₗₐₛₜ | |
|---|---|---|---|---|---|---|
| representation | Cₘₐₓ | Tₘₐₓ | (mL/ | (mL/ | (ng · | T_{1/2,app} |
| (SEQ ID NO:) | (ng/ml) | (hr) | hr/kg) | kg) | hr/mL) | (hr) |
| FM9-4 + Fc_hole | 8421.37 | 24 | 1.30 | 69.31 | 750497.02 | 36.84 |
| (SEQ ID NOs: 101 | | | | | | |
| and 47) | | | | | | |
| FM9-6 + Fc_hole | 7581.92 | 24 | 0.91 | 111.02 | 939308.72 | 84.28 |
| (SEQ ID NOs: 103 | | | | | | |
| and 47) | | | | | | |
| FM11-4 + Fc_hole | 7200.02 | 24 | 1.41 | 82.63 | 690213.08 | 40.61 |
| (SEQ ID NOs: 107 | | | | | | |
| and 47) | | | | | | |
| FM11-6 + Fc_hole | 1547.77 | 24 | 8.43 | 314.10 | 116433.94 | 25.84 |
| (SEQ ID NOs: 109 | | | | | | |
| and 47) | | | | | | |

### Example 4: Evaluation of Anti-Obesity Effect by Administration of Long-Acting GDF15 Fusion Protein (Dimer) Alone and in Combination with Semaglutide in Diet-Induced Obese (DIO) Mice

Diet-induced obese (DIO) mice, obtained by feeding mice with high-fat food, are characterized by obesity, hyperglycemia, and insulin resistance. Diet-induced obese mice (Taconic, USA) resulting from feeding C57BL/6N mice with high-fat food (60 kcal % fat, Research Diets, Cat #D12492, USA) for 8 weeks were purchased from Raon Bio (Animal Inc., Republic of Korea). The diet-induced obese mice were additionally fed with 60% high-fat food for 5 weeks after arrival, and then used in the present test. On the day before drug treatment, the mice were grouped based on the average body weight of individual mice (n=6 per group), after which 10 and 30 nmol/kg of FM9-6+Fc_hole alone, 10 and 30 nmol/kg of semaglutide (long-acting GLP-1 derivative, SEQ ID NO: 114) alone as a control drug, and FM9-6+Fc_hole and semaglutide were administered subcutaneously every 2 days (Q2D) for a total of 8 weeks in a combination of 10+10 and 30+30 nmol/kg. The semaglutide that was used in the test was Ozempic® (semaglutide) from Novo NorDisk. Here, for vehicle treatment, DPBS (Dulbecco's phosphate-buffered saline, Gibco, USA) was subcutaneously administered every 2 days (Q2D). Body weight was measured every 2 days from the first day of drug treatment to day 54, and the results thereof are shown in Table 4 below.

TABLE 4

| | Dose | Change in body weight (%) vs. day 0 | |
|---|---|---|---|
| Group | (nmol/kg) | Maximum efficacy | Efficacy on Day 54 |
| DIO Vehicle | — | — | 6.52 |
| FM9-6 + Fc_hole | 10 | −34.57 (On Day 54)**** | −34.57**** |
| FM9-6 + Fc_hole | 30 | −35.07 (On Day 54)**** | −35.07**** |
| FM9-6 + Fc_hole + | 10 + 10 | −44.05 (On Day 54)**** | −44.05**** |
| Semaglutide | | | |
| FM9-6 + Fc_hole + | 30 + 30 | −50.03 (On Day 54)**** | −50.03**** |
| Semaglutide | | | |
| Semaglutide | 10 | −26.71 (On Day 30)**** | −17.34**** |
| Semaglutide | 30 | −28.13 (On Day 21)**** | −13.40**** |

****p < 0.0001 vs. obese control (DIO vehicle) (Two-way ANOVA).

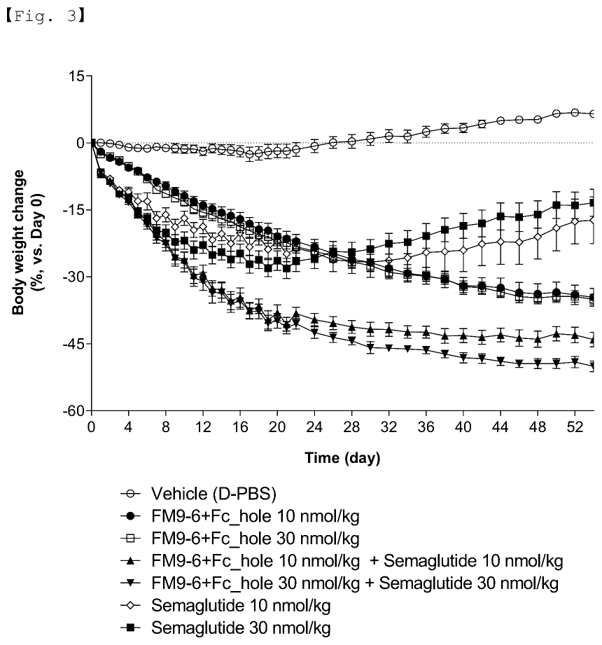

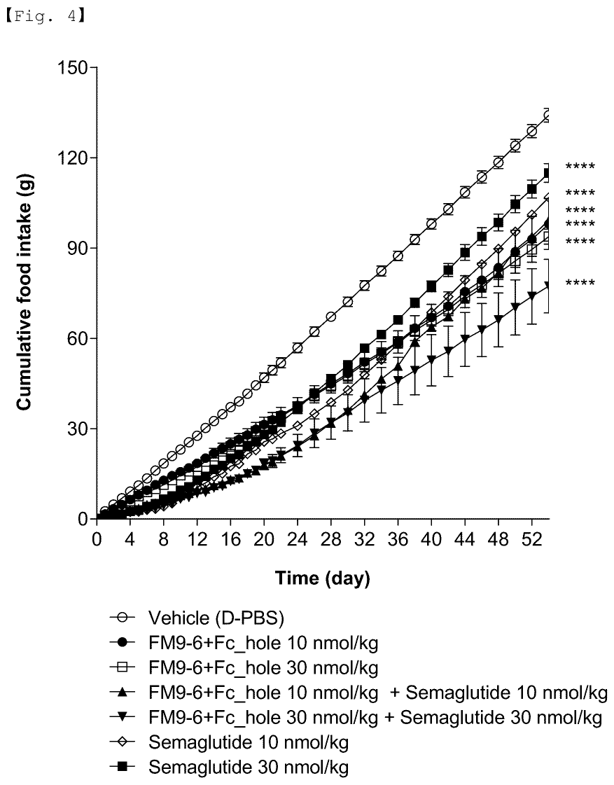

Consequently, additive effects on weight loss and Food intake reduction were confirmed in the groups administered with FM9-6+Fc_hole and semaglutide in combination compared to the groups administered with FM9-6+Fc_hole alone or semaglutide alone (FIGS. 3 and 4).

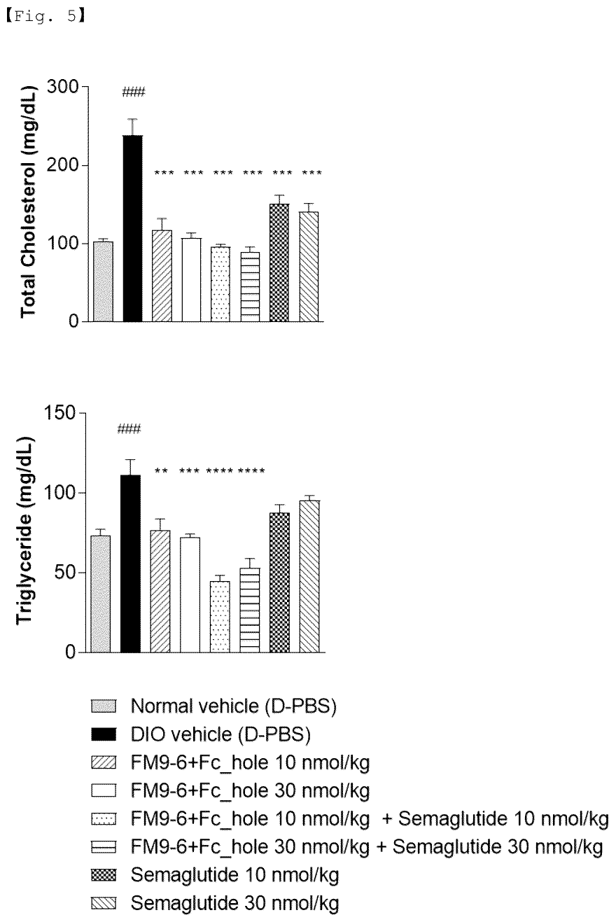

Moreover, when FM9-6+Fc_hole and semaglutide were administered in combination, an effect equal to or greater than the maximum efficacy of administration of each material alone was exhibited, and levels of total cholesterol and triglyceride were decreased normal range for mice. (FIG. 5).

### Example 5: Evaluation of Anti-Obesity Effect by Repeated Administration of Optimized Long-Acting GDF15 Fusion Protein (Dimer) Alone and in Combination with Semaglutide in Ob/Ob Mice

The ob/ob mouse has a mutation in the leptin gene resulting in leptin deficiency, and are characterized by hyperglycemia, insulin resistance, overeating, and obesity. 5-week-old male ob/ob mice (Jackson Laboratory, USA) were purchased from Raon Bio (Animal Inc., Republic of Korea). The ob/ob mice were acclimatized with normal food (Teklad Certified Irradiated Global 18% Protein Rodent Diet, 2918C, Harlan Co., USA) for 4 weeks, and drug treatment was started at 9 weeks of age. On the day before drug treatment, the mice were grouped based on the body weight of individual mice and random blood glucose levels determined by sampling at the tail vein (n=6 per group). Thereafter, 1 and 10 nmol/kg of FM9-6+Fc_hole alone, 10 and 30 nmol/kg of semaglutide alone, and FM9-6+Fc_hole and semaglutide at doses of 1+10 and 10+10 nmol/kg in combination were subcutaneously administered thereto a total of 10 times every 3 days (Q3D), and body weight and food intake were measured every day or every 3 days during the experimental period (29 days). Here, DPBS (Dulbecco's phosphate-buffered saline, Gibco, USA) was administered for vehicle treatment. The results thereof are shown in Table 5 below.

TABLE 5

| | Dose | Change in body weight (%) vs. ob/ob control | |
|---|---|---|---|
| Group | (nmol/kg) | Maximum efficacy | Efficacy on Day 29 |
| ob/ob Vehicle | — | — | — |
| FM9-6 + Fc_hole | 1 | −21.49 (On Day 29)**** | −21.49**** |
| FM9-6 + Fc_hole | 10 | −24.87 (On Day 29)**** | −24.87 **** |
| FM9-6 + Fc_hole + | 1 + 10 | −27.68 (On Day 28)**** | −26.87**** |
| Semaglutide | | | |
| FM9-6 + Fc_hole + | 10 + 10 | −28.76 (On Day 28)**** | −28.36**** |
| Semaglutide | | | |
| Semaglutide | 10 | −10.04 (On Day 25)*** | −6.88* |
| Semaglutide | 30 | −11.38 (On Day 25)**** | −9.92**** |

*p < 0.05,
***p < 0. 001, and
****p < 0.0001 vs. obese control (ob/ob vehicle) (Two-way ANOVA).

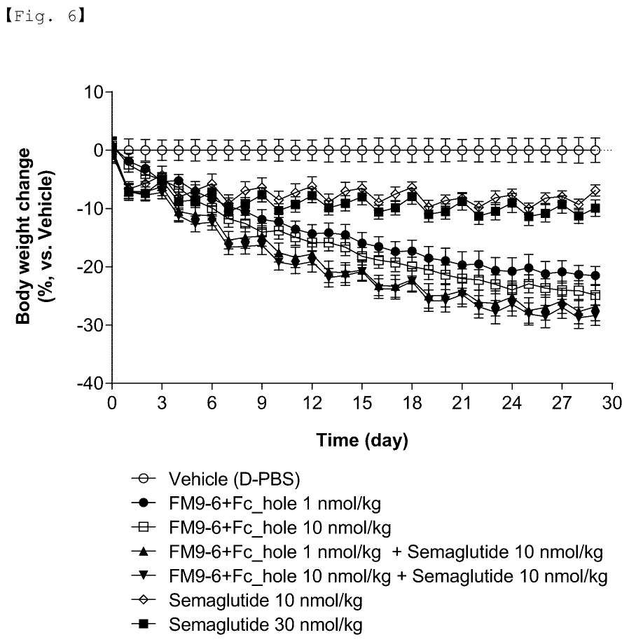

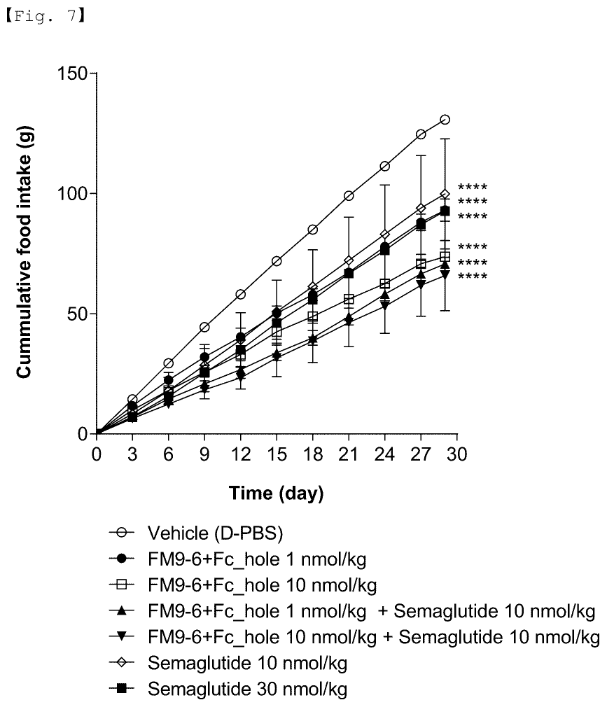

Consequently, the effects of weight loss and food intake reduction were found to be superior in the groups administered with FM9-6+Fc_hole in and semaglutide combination compared to the groups administered with FM9-6+Fc_hole alone, and also, a significant weight loss effect equal to or greater than the maximum efficacy for the group administered with semaglutide alone was confirmed. In the groups administered with FM9-6+Fc_hole and semaglutide in combination, an equivalent weight loss effect was exhibited between the group co-administered with FM9-6+Fc_hole at a low dose (1 nmol/kg) and the group co-administered with FM9-6+Fc_hole at a high dose (10 nmol/kg) (FIGS. 6 and 7).

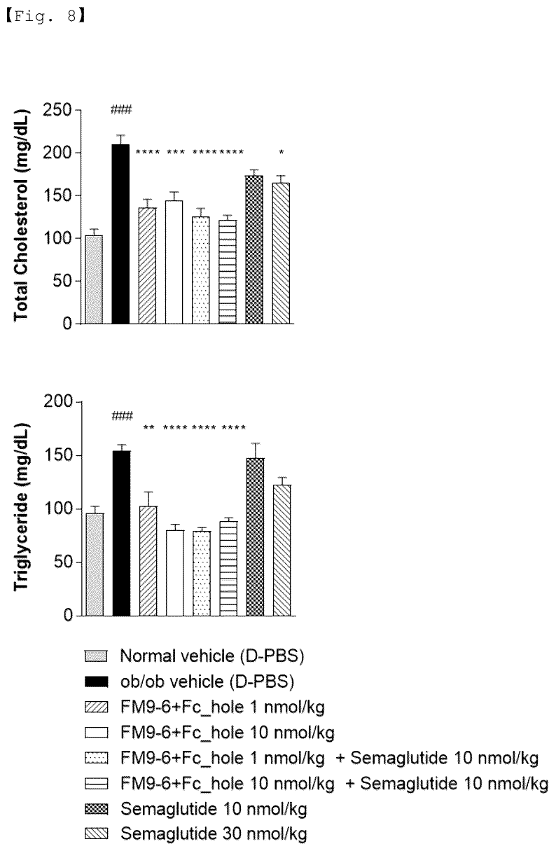

In addition, when FM9-6+Fc_hole and semaglutide were administered in combination, blood cholesterol and triglyceride levels were significantly decreased compared to the group administered with semaglutide alone, and the efficacy of reduction of blood lipid levels upon administration of FM9-6+Fc_hole at a high dose (10 nmol/kg) alone was also observed in the group administered with FM9-6+Fc_hole at a low dose and semaglutide in combination (1+10 nmol/kg) (FIG. 8).

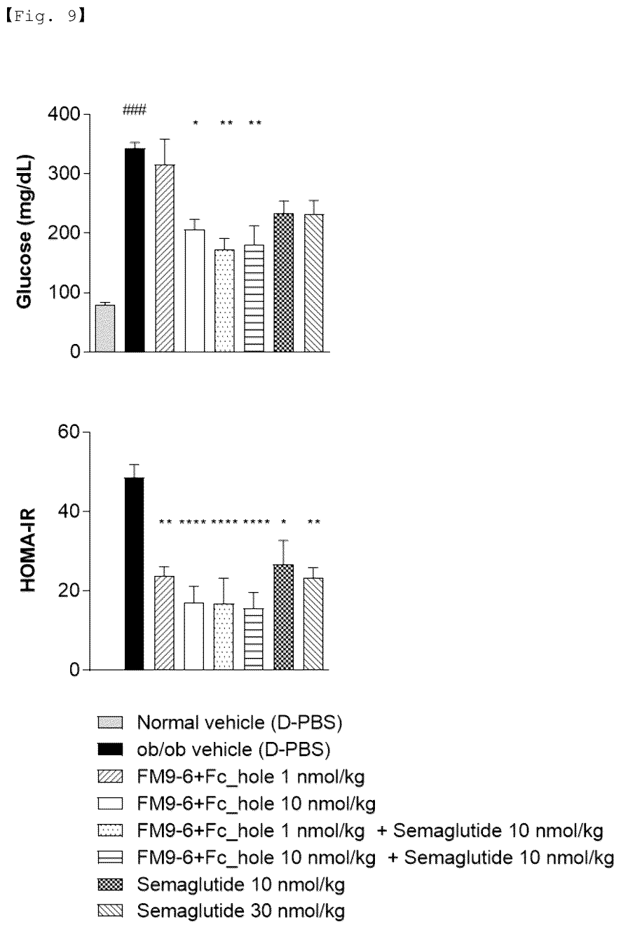

Moreover, the blood-glucose-lowering effect and insulin resistance improvement effect were found to be superior in the group administered with FM9-6+Fc_hole at a low dose and semaglutide in combination (1+10 nmol/kg) compared to the group administered with FM9-6+Fc_hole (1 nmol/kg) alone or semaglutide (10 nmol/kg) alone, and the efficacy upon administration of FM9-6+Fc_hole at a high dose (10 nmol/kg) alone was also observed in the group co-administered with FM9-6+Fc_hole at a low dose (FIG. 9).

### Example 6: Evaluation of Anti-Obesity and Metabolic Parameter Improvement Effects by Repeated Administration of Optimized Long-Acting GDF15 Fusion Protein (Dimer) Alone and in Combination with Large Dose of Semaglutide in Ob/Ob Mice

5-week-old male ob/ob mice (Jackson Laboratory, USA) were purchased from Raon Bio (Animal Inc., Republic of Korea). The ob/ob mice were acclimatized with normal chow or normal chow diet (Teklad Certified Irradiated Global 18% Protein Rodent Diet, 2918C, Harlan Co., USA) for 5 weeks, and drug treatment was started at 10 weeks of age. On the day before drug treatment, the mice were grouped based on the body weight of individual mice and random blood glucose levels determined by sampling at the tail vein (n=6 per group). Thereafter, 1 nmol/kg of FM9-6+Fc_hole alone, 30 nmol/kg of semaglutide alone, and FM9-6+Fc_hole and semaglutide in combination (1+30 nmol/kg) were subcutaneously administered thereto a total of 9 times every 3 days (Q3D), and body weight, food intake, and non-fasting blood glucose were measured every day or every 3 days during the experimental period (24 days). Here, DPBS (Dulbecco's phosphate-buffered saline, Gibco, USA) was administered for vehicle treatment. The results thereof are shown in Table 6 below.

TABLE 6

| | | | Mean of random |
|---|---|---|---|
| | | Change in body weight | blood glucose |
| | Dose | (%) vs. ob/ob control | levels (mg/dL) |
| Group | (nmol/kg) | Efficacy on Day 24 | Efficacy on Day 24 |
| ob/ob Vehicle | — | — | 204.3 |
| FM9-6 + Fc_hole | 1 | −23.37**** | 116.2**** |
| Semaglutide | 30 | −10.08**** | 155.7**** |
| FM9-6 + Fc_hole + | 1 + 30 | −36.40**** | 88.7**** |
| Semaglutide | | | |

****p < 0.0001 vs. obese control (ob/ob vehicle) (Two-way ANOVA).

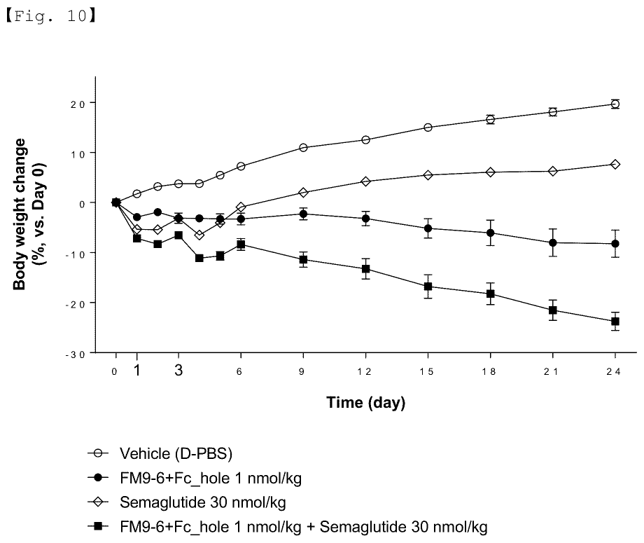

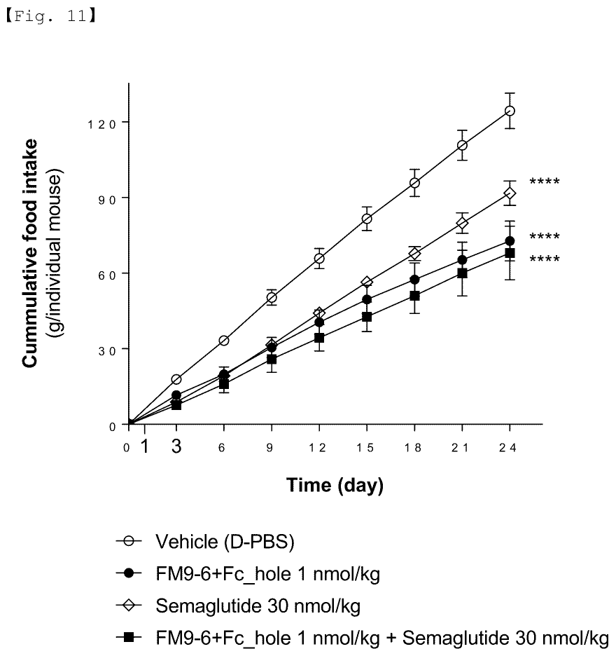

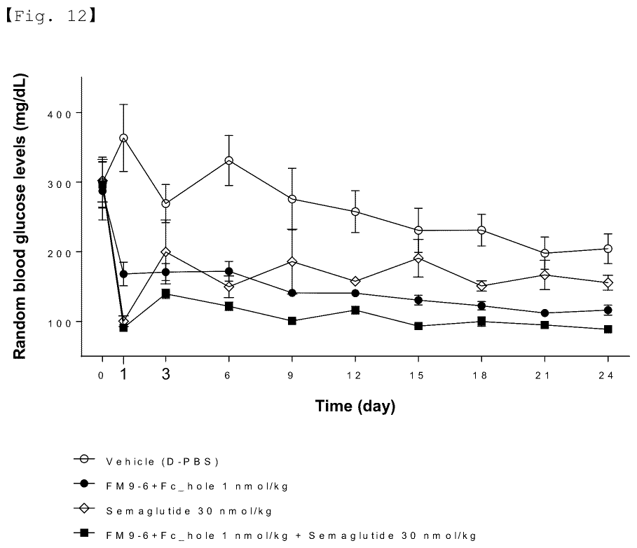

Consequently, the weight loss, food intake reduction, and blood-glucose-lowering effects were found to be superior in the group administered with FM9-6+Fc_hole and semaglutide in combination the compared to group administered with FM9-6+Fc_hole alone, and significant weight loss and blood-glucose-lowering effects equal to or greater than the maximum efficacy for the group administered with semaglutide alone were confirmed. Moreover, when compared with the group administered with FM9-6+Fc_hole alone or semaglutide alone, additive effects on weight loss or food intake reduction were confirmed in the group administered with FM9-6+Fc_hole and semaglutide in combination (FIGS. 10, 11 and 12).

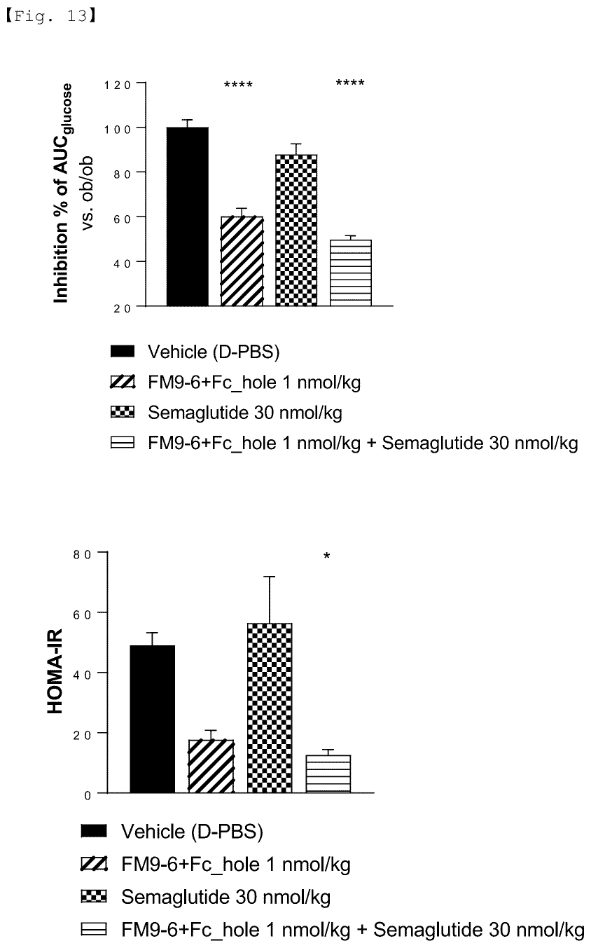

In the oral tolerance test and values of homeostasis model assessment of insulin resistance (HOMA-IR) index performed at the end of repeated administration, the group administered with FM9-6+Fc_hole (1 nmol/kg) and semaglutide (30 nmol/kg) in combination exhibited superior metabolic parameter improvement effects compared to the group administered with FM9-6+Fc_hole alone or semaglutide alone (FIG. 13).

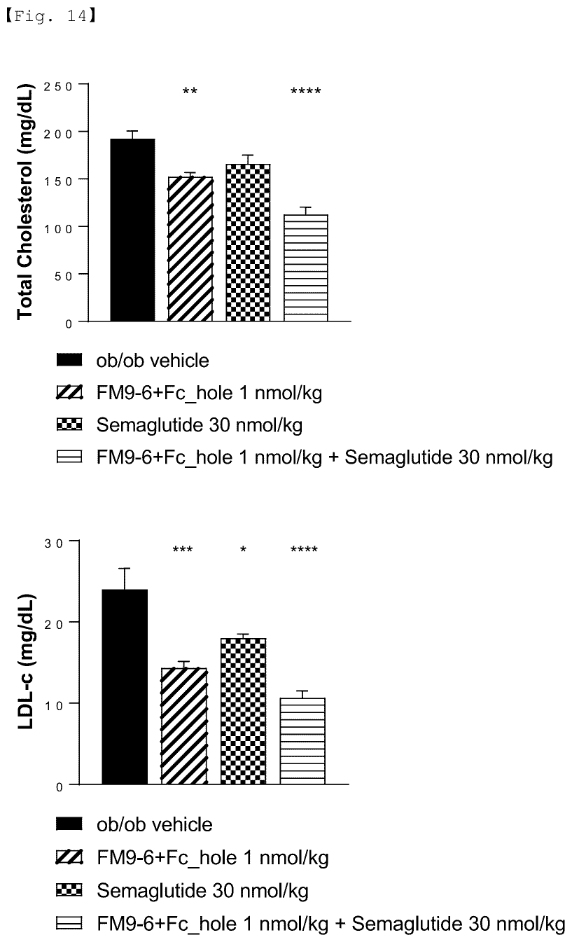

In addition, when FM9-6+Fc_hole and semaglutide were administered in combination, great reductions in total cholesterol and LDL-c (low-density lipoprotein-cholesterol) were observed compared to the group administered with semaglutide alone (FIG. 14).

### Example 7: Evaluation of Anti-Obesity Effect by Administration of Long-Acting GDF15 Fusion Protein (Dimer) Alone and in Combination with Semaglutide in Diet-Induced Obese (DIO) Mice

Diet-induced obese (DIO) mice, obtained by feeding mice with high-fat diet, are characterized by obesity, hyperglycemia, and insulin resistance. Diet-induced obese mice (Taconic, USA) resulting from feeding C57BL/6N mice with high-fat food (60 kcal % fat, Research Diets, Cat #D12492, USA) for 8 weeks were purchased from Raon Bio (Animal Inc., Republic of Korea). The diet-induced obese mice were additionally fed with 60% high-fat food for 5 weeks after arrival, and then used in the present test. On the day before drug treatment, the mice were grouped based on the average body weight of individual mice (n=6 per group), after which 0.1 and 1 nmol/kg of FM9-6+Fc_hole alone, 10 nmol/kg of semaglutide (long-acting GLP-1 derivative, SEQ ID NO: 114) and 10 nmol/kg of tirzepatide (long-acting GLP-1/GIP derivative, SEQ ID NO: 117) alone as control drugs, FM9-6+Fc_hole and semaglutide at doses of 0.1+10 and 1+10 nmol/kg in combination, and FM9-6+Fc_hole and tirzepatide at doses of 1+10 nmol/kg in combination were subcutaneously administered thereto every 3 days (Q3D) for a total of 4 weeks. The semaglutide that was used in the test was Ozempic® (semaglutide) from Novo NorDisk, and the tirzepatide that was used was a material synthesized by MedChemExpress (HY-P1731B, USA). Here, DPBS (Dulbecco's phosphate-buffered saline, Gibco, USA) was administered for vehicle treatment, and, during the experimental period (26 days), body weight was measured every day and food intake was measured every 3 days. The results thereof are shown in Table 7 below.

TABLE 7

| | Dose | Change in body weight (%) vs. day 0 | |
|---|---|---|---|
| Group | (nmol/kg) | Maximum efficacy | Efficacy on Day 26 |
| DIO Vehicle | — | — | — |
| FM9-6 + Fc_hole | 0.1 | −6.75 (On Day 25)** | −6.68** |
| FM9-6 + Fc_hole | 1 | −16.33 (On Day 23)**** | −16.29**** |
| Semaglutide | 10 | −18.95 (On Day 19)**** | −17.69**** |
| FM9-6 + Fc_hole + | 0.1 + 10 | −27.92 (On Day 25)**** | −26.06**** |
| Semaglutide | | | |
| FM9-6 + Fc_hole + | 1 + 10 | −31.51 (On Day 25)**** | −29.90**** |
| Semaglutide | | | |
| Tirzepatide | 10 | −24.79 (On Day 25)**** | −24.60**** |
| FM9-6 + Fc_hole + | 1 + 10 | −38.71 (On Day 26)**** | −38.71**** |
| Tirzepatide | | | |

**p < 0.01, and
****p < 0.0001 vs. obese control (DIO vehicle) (Two-way ANOVA).

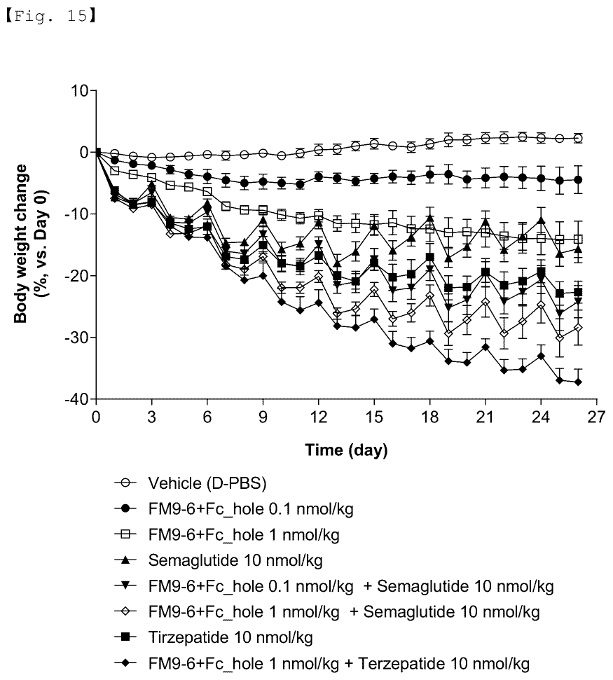

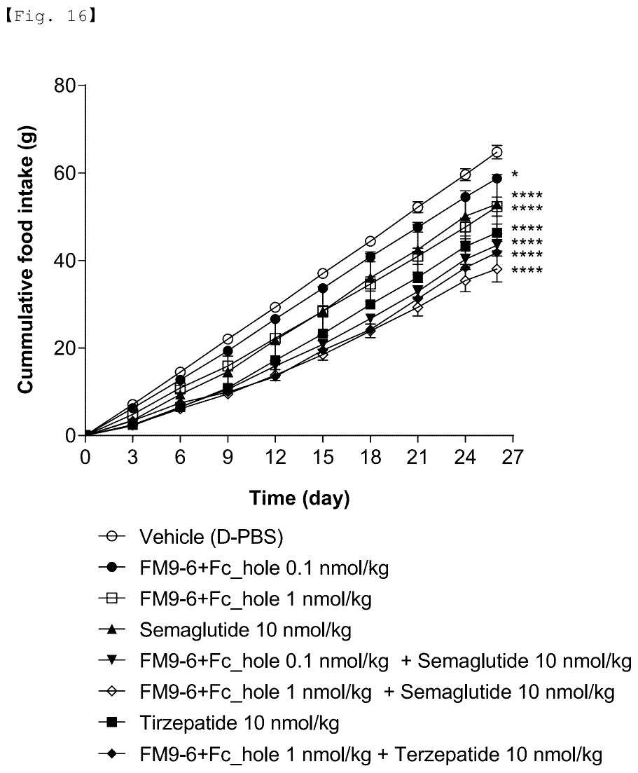

Consequently, additive effects on weight loss and food intake reduction were confirmed in the groups administered with FM9-6+Fc_hole and semaglutide in combination compared to the groups administered with FM9-6+Fc_hole alone or semaglutide alone. The group administered with FM9-6+Fc_hole at a low dose and semaglutide in combination (0.1+10 nmol/kg) exhibited a weight loss effect equivalent to the group administered with tirzepatide alone (10 nmol/kg), and the group administered with FM9-6+Fc_hole at a high dose and semaglutide in combination (1+10 nmol/kg) showed or more potent than the group administered with tirzepatide alone. In addition, when FM9-6+Fc_hole and tirzepatide were administered in combination, an additive effect on weight loss was confirmed compared to the groups administered with FM9-6+Fc_hole alone or tirzepatide alone (FIGS. 15 and 16).

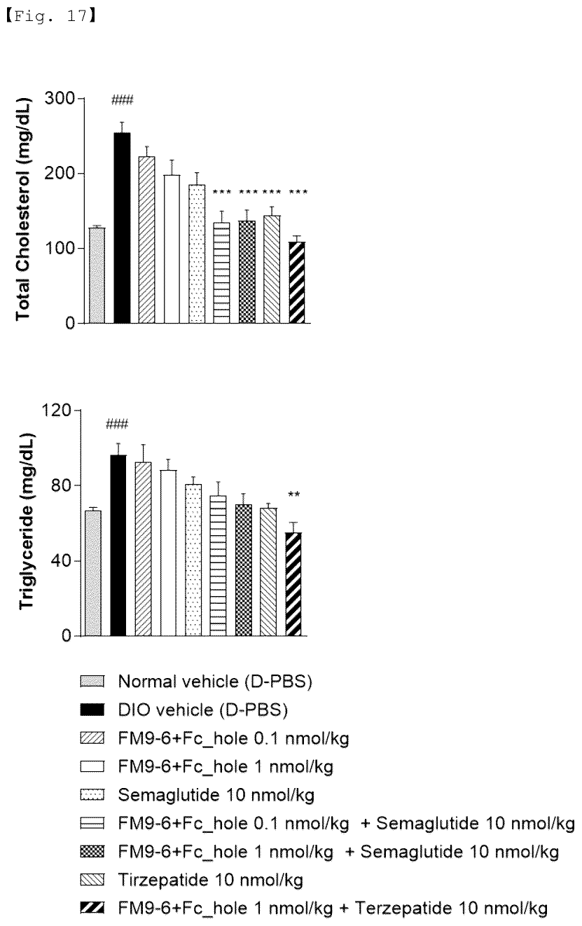

Moreover, in the groups administered with FM9-6+Fc_hole and semaglutide in combination, reductions in total cholesterol levels superior to the maximum efficacy of administration of FM9-6+Fc_hole alone or semaglutide alone were exhibited, which was equivalent to the group administered with tirzepatide alone, resulting in reduction to the normal range. Also, when FM9-6+Fc_hole and semaglutide were administered in combination, levels of triglyceride were decreased to a normal range for mice, and in the group administered with FM9-6+Fc_hole and tirzepatide in combination, a significant decrease in triglyceride level was confirmed (FIG. 17).

### Example 8: Evaluation of Inhibitory Effect on Gastric Emptying by Administration of Long-Acting GDF15 Fusion Protein (Dimer) Alone and in Combination with Semaglutide in Normal Rats

A GLP-1 receptor agonist is characterized by strong inhibition of gastric emptying (Can et al., Int. J. Obes. 2014; 38:784-793), and this inhibition effect has also been reported for native GDF15 depending on the dose (Borner et al., Cell Reports 2020; 31:107543). Therefore, this example was performed to investigate the inhibitory effect on gastric emptying when the novel long-acting GDF15 fusion protein was administered at the doses equal to the effective dose, higher than the maximum effective dose, or in combination with a GLP-1 receptor agonist to rats. 6-week-old male SD rats (Orient Bio, Republic of Korea) were purchased from Hallym Lab. Animal Inc., Republic of Korea. Animals were acclimated for 7 days. On the day of drug administration, the rats were grouped based on the average body weight of individual rats (n=5 per group) and fasted for at least 6 hours before oral administration of acetaminophen. Then, 0.1, 1, and 10 nmol/kg of FM9-6+Fc_hole, 10 nmol/kg of semaglutide (long-acting GLP-1 derivative, SEQ ID NO: 114), and combination of FM9-6+Fc_hole and semaglutide (1+10 nmol/kg) were subcutaneously administered. The semaglutide was Ozempic® (semaglutide) from Novo NorDisk. As a negative control drug, D-PBS (normal vehicle) was subcutaneously administered, and as a positive control drug, 30 mg/kg of loperamide was orally administered 45 minutes before acetaminophen administration. On 3, 24 and 72 hours after a single dose of the test articles, acetaminophen was orally administered at 100 mg/kg to rats, and blood samples were collected before and at 30, 60, 90, 120 and 150 minutes after acetaminophen administration. The plasma concentration of acetaminophen was analyzed, and the pharmacokinetic profiles and the systemic exposures of acetaminophen were compared to evaluate the inhibitory effect on gastric emptying among the groups. The results are shown in Table 8 below.

TABLE 8

| | | | AUCₗₐₛₜ of acetaminophen | | |
|---|---|---|---|---|---|
| | | | (μg · min/mL) | | |
| Group | Dose | | 3 hr | 24 hr | 72 hr |
| Vehicle (DPBS) | — | | 2252.13 | 2812.14 | 3893.85 |
| FM9-6 + Fc_hole | 0.1 | nmol/kg | 1873.02 | 2652.42 | 3054.78 |
| FM9-6 + Fc_hole | 1 | nmol/kg | 2615.40 | 1878.30 | 3526.86 |
| FM9-6 + Fc_hole | 10 | nmol/kg | 1497.84 | 2385.36 | 3100.59 |
| Semaglutide | 10 | nmol/kg | 266.43*** | 2577.72 | 3354.48 |
| FM9-6 + Fc_hole + | 1 nmol/kg + | | 422.61*** | 2029.89 | 3226.65 |
| Semaglutide | 10 nmol/kg | | | | |
| Loperamide | 30 | mg/kg | 1565.13 | 761.04** | 996.36*** |

**p < 0.01, and
***p < 0.001 vs. negative control (normal vehicle) (One-way ANOVA)

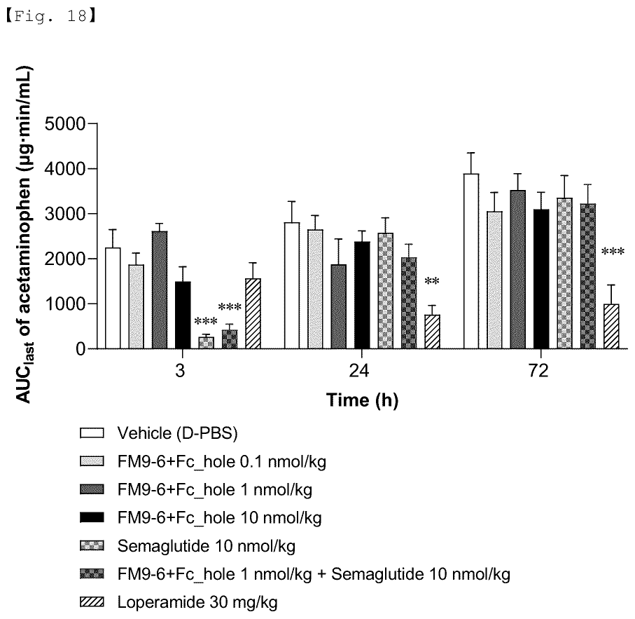

Consequently, in semaglutide group, the systemic exposure of acetaminophen at 3 hours after administration was significantly low compared to negative control group, indicating the inhibition of gastric emptying. At 24 and 72 hours after semaglutide administration, the systemic exposures of acetaminophen were mostly comparable to negative control group, indicating that the gastric emptying was restored. In 0.1, 1, and 10 nmol/kg of FM9-6+Fc_hole groups, there was no statistical difference in systemic exposures of acetaminophen at 3, 24, and 72 hours after administration compared to negative control group, indicating that FM9-6+Fc_hole did not affect gastric emptying at the tested dose range. In addition, no significant difference in systemic exposure of acetaminophen was observed in combination group of FM9-6+Fc_hole and semaglutide compared to semaglutide group, indicating that the combination of FM9-6+Fc_hole and semaglutide did not provide additive effect on inhibition of gastric emptying (FIG. 18).

### INDUSTRIAL APPLICABILITY

According to the present invention, a composition including a GDF15 variant, a long-acting GDF15 fusion protein, or a long-acting GDF15 fusion protein dimer and a GLP-1 receptor agonist has no effect on gastric emptying, and is effective at preventing or treating diabetes, obesity, dyslipidemia, or metabolic syndrome.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereof.

What is claimed is:

1. A pharmaceutical composition for preventing or treating diabetes, obesity, dyslipidemia, or metabolic syndrome by administering in combination with a GLP-1 (glucagon-like peptide-1) receptor agonist, comprising a GDF15 (growth differentiation factor-15) variant represented by Formula (I) below, a long-acting GDF15 fusion protein, or a long-acting GDF15 fusion protein dimer as an active ingredient:
$$N - terminal extension domain - core domain \qquad Formula ( I )$$
in Formula (I),
the N-terminal extension domain is a polypeptide comprising any one amino acid sequence selected from among SEQ ID NOS: 3 to 5; and
the core domain is a polypeptide comprising an amino acid sequence of SEQ ID NO: 20, or is a polypeptide in which any one selected from the group consisting of a 15^{th} amino acid, 50^{th} amino acid, 58^{th} amino acid, 97^{th} amino acid, and combinations thereof in the amino acid sequence of SEQ ID NO: 20 is substituted with another amino acid,
in which arginine (R), which is the 15^{th} amino acid, is substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),
asparagine (N), which is the 50^{th} amino acid, is substituted with alanine, arginine (R), aspartic acid, cysteine, glutamic acid, glutamine, glycine, histidine, isoleucine, leucine, lysine, methionine, phenylalanine, proline, serine, threonine, tryptophan, tyrosine, or valine,
serine (S), which is the 58^{th} amino acid, is substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or
aspartic acid (D), which is the 97^{th} amino acid, is substituted with alanine, arginine, asparagine, cysteine, glutamic acid, glutamine, glycine, histidine, isoleucine, leucine, lysine, methionine, phenylalanine, proline, serine, threonine, tryptophan, tyrosine, or valine.

2. The pharmaceutical composition according to claim 1, wherein the core domain comprises any one mutation selected from the group consisting of mutations (1) to (6) below:
(1) arginine (R), which is the 15^{th} amino acid in the amino acid sequence of SEQ ID NO: 20, is substituted with asparagine (N);
(2) asparagine (N), which is the 50^{th} amino acid in the amino acid sequence of SEQ ID NO: 20, is substituted with leucine (L);
(3) serine (S), which is the 58^{th} amino acid in the amino acid sequence of SEQ ID NO: 20, is substituted with lysine (K), arginine (R), asparagine (N), aspartic acid (D), glutamic acid (E), cysteine (C), or leucine (L);
(4) aspartic acid (D), which is the 97^{th} amino acid in the amino acid sequence of SEQ ID NO: 20, is substituted with leucine (L);
(5) asparagine (N), which is the 50^{th} amino acid, and aspartic acid (D), which is the 97^{th} amino acid, in the amino acid sequence of SEQ ID NO: 20, are substituted with cysteine (C) or serine (S); and
(6) arginine (R), which is the 15^{th} amino acid in the amino acid sequence of SEQ ID NO: 20, is substituted with asparagine (N), and serine (S), which is the 58^{th} amino acid, is substituted with lysine (K) or arginine (R).

3. The pharmaceutical composition according to claim 2, wherein the core domain comprises any one amino acid sequence selected from among SEQ ID NOS: 6 to 19.

4. The pharmaceutical composition according to claim 1, wherein the GDF15 variant comprises an N-terminal extension domain comprising an amino acid sequence set forth in SEQ ID NO: 3 and a core domain comprising an amino acid sequence set forth in SEQ ID NO: 8, 9, or 20.

5. The pharmaceutical composition according to claim 1, wherein the GDF15 variant comprises an N-terminal extension domain comprising an amino acid sequence set forth in SEQ ID NO: 4 and a core domain comprising an amino acid sequence set forth in SEQ ID NO: 8, 9, or 20.

6. The pharmaceutical composition according to claim 1, wherein the GDF15 variant comprises an N-terminal extension domain comprising an amino acid sequence set forth in SEQ ID NO: 5 and a core domain comprising any one amino acid sequence selected from among SEQ ID NOs: 6 to 19.

7. The pharmaceutical composition according to claim 1, wherein the GDF15 variant comprises any one amino acid sequence selected from among SEQ ID NOs: 21 to 39.

8. The pharmaceutical composition according to claim 1, wherein the long-acting GDF15 fusion protein is configured such that the GDF15 variant and a human IgG Fc or a variant thereof are bound to each other.

9. The pharmaceutical composition according to claim 1, wherein the long-acting GDF15 fusion protein dimer comprises two long-acting GDF15 fusion proteins.

10. The pharmaceutical composition according to claim 8, wherein the IgG Fc or the variant thereof comprises:
a first polypeptide comprising an IgG1 Fc sequence, the IgG1 Fc sequence comprising a CH3 sequence comprising at least one engineered protuberance; and
a second polypeptide comprising an IgG1 Fc sequence, the IgG1 Fc sequence comprising a CH3 sequence comprising at least one engineered cavity,
wherein the first polypeptide and the second polypeptide are heterodimerized via positioning of the protuberance of the first polypeptide into the cavity of the second polypeptide.

11. The pharmaceutical composition according to claim 8, wherein a C-terminus of a first polypeptide or a C-terminus of a second polypeptide of the IgG Fc or the variant thereof is bound to an N-terminus of the GDF15 variant.

12. The pharmaceutical composition according to claim 8, wherein the GDF15 variant and the IgG Fc or the variant thereof are bound via a linker.

13. The pharmaceutical composition according to claim 12, wherein the linker is a peptide comprising glycine, serine, alanine, lysine and glutamic acid residues and composed of 10 to 50 amino acid residues.

14. The pharmaceutical composition according to claim 12, wherein the linker is GGGGGGGGSGGGGSGGGGSGGGGS (SEQ ID NO: 48), GSGGGGSGGGGSGGGGS (SEQ ID NO: 92), GSGGGGSGGGGSGGGGSGGGGSGGGGS (SEQ ID NO: 93), GSGGGGSGGGGSGGGGSGGGGSGGGGSGGGGSGGGGS (SEQ ID NO: 94), GSEEEAEEEAEEEAEEEAEEEAEEEA (SEQ ID NO: 95), GSGGSSPTPTPTPTPTPTPTPTPTPT (SEQ ID NO: 96), or GSEAAAKEAAAKEAAAKEAAAKEAAAK (SEQ ID NO: 97).

15. The pharmaceutical composition according to claim 10, wherein the first polypeptide comprises any one amino acid sequence selected from among SEQ ID NOs: 42, 44, and 46.

16. The pharmaceutical composition according to claim 10, wherein the second polypeptide comprises any one amino acid sequence selected from among SEQ ID NOs: 43, 45, and 47.

17. The pharmaceutical composition according to claim 1, wherein the GDF15 variant comprises at least one N-linked glycan (N-linked glycan).

18. The pharmaceutical composition according to claim 1, wherein the long-acting GDF15 fusion protein comprises i) a GDF15 variant comprising any one amino acid sequence selected from among SEQ ID NOs: 21 to 39, ii) a first polypeptide comprising any one amino acid sequence selected from among SEQ ID NOs: 42, 44, and 46, and iii) a second polypeptide comprising any one amino acid sequence selected from among SEQ ID NOs: 43, 45, and 47.

19. The pharmaceutical composition according to claim 1, wherein the GLP-1 receptor agonist comprises GLP-1, a fragment thereof, or an analogue thereof.

20. The pharmaceutical composition according to claim 1, wherein the GLP-1 receptor agonist is at least one selected from the group consisting of exenatide, liraglutide, lixisenatide, albiglutide, dulaglutide, semaglutide, tirzepatide, cotadutide, and taspoglutide.

21. The pharmaceutical composition according to claim 1, wherein the GDF15 variant, the long-acting GDF15 fusion protein, or the long-acting GDF15 fusion protein dimer and the GLP-1 receptor agonist are provided in a complex formulation.

22. The pharmaceutical composition according to claim 1, wherein the GDF15 variant, the long-acting GDF15 fusion protein, or the long-acting GDF15 fusion protein dimer and the GLP-1 receptor agonist are administered simultaneously or sequentially.

23. A pharmaceutical composition for preventing or treating diabetes, obesity, dyslipidemia, or metabolic syndrome by administering in combination with a GLP-1 (glucagon-like peptide-1) receptor agonist, comprising a complex comprising a growth differentiation factor-15 (GDF15) variant and an IgG Fc, said complex being represented by the following formula (II):
IgG Fc-(L)ₘ-N-terminal extension domain-core domain (II)
wherein m is an integer of 0 or 1,
L is a linker selected from the group consisting of SEQ ID NOs: 48, 92, 93, 94, 95, 96, and 97,
IgG Fc comprises the amino acid sequence of SEQ ID NOs: 42, 44, or 46, and
N-terminal extension domain and core domain are as defined in claim 1.

24. The pharmaceutical composition according to claim 23, wherein the N-terminal extension domain-core domain comprises any one amino acid sequence selected from the group consisting of SEQ ID NOS: 21 to 39.

25. The pharmaceutical composition according to claim 23, wherein the complex comprises any one amino acid sequence selected from the group consisting of SEQ ID NOS: 50-91 and 98-109.


SEQUENCE LISTING (118 sequences)

SEQ ID NO: 1 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 106 aa, Artificial Sequence
  Core Domain (R21X, N56X, S64X, D103X)
  misc_feature (15)..(15): Xaa can be any naturally occurring amino acid
  misc_feature (50)..(50): Xaa can be any naturally occurring amino acid
  misc_feature (58)..(58): Xaa can be any naturally occurring amino acid
  misc_feature (97)..(97): Xaa can be any naturally occurring amino acid
CPLGPGRCCRLHTVXASLEDLGWADWVLSPREVQVTMCIGACPSQFRAAXMHAQIKTXLHRLKPDTVPAPCCVPASYNPMVLIQKTDTGVSLQTYDXLLAKDCHCI

SEQ ID NO: 3 - protein, 5 aa, Artificial Sequence
  N-terminal extension domain I
WSNST

SEQ ID NO: 4 - skipped

SEQ ID NO: 5 - protein, 4 aa, Artificial Sequence
  N-terminal extension domain III
NGDH

SEQ ID NO: 6 - protein, 106 aa, Artificial Sequence
  Core Domain(R21N)
CPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 7 - protein, 106 aa, Artificial Sequence
  Core Domain(N56L)
CPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 8 - protein, 106 aa, Artificial Sequence
  Core Domain(S64K)
CPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 9 - protein, 106 aa, Artificial Sequence
  Core Domain(S64R)
CPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 10 - protein, 106 aa, Artificial Sequence
  Core Domain(S64N)
CPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 11 - protein, 106 aa, Artificial Sequence
  Core Domain(S64D)
CPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 12 - protein, 106 aa, Artificial Sequence
  Core Domain(S64E)
CPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 13 - protein, 106 aa, Artificial Sequence
  Core Domain(S64C)
CPLGPGRCCRLHTVRASLEDLGWADWVLSPREVQVTMCIGACPSQFRAANMHAQIKTCLHRLKPDTVPAPCCVPASYNPMVLIQKTDTGVSLQTYDDLLAKDCHCI

SEQ ID NO: 14 - protein, 106 aa, Artificial Sequence
  Core Domain(S64L)
CPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 15 - protein, 106 aa, Artificial Sequence
  Core Domain(D103L)
CPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 16 - protein, 106 aa, Artificial Sequence
  Core Domain(N56C, D103C)
CPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 17 - protein, 106 aa, Artificial Sequence
  Core Domain(N56S, D103S)
CPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 18 - protein, 106 aa, Artificial Sequence
  Core Domain(R21N, S64R)
CPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 19 - protein, 106 aa, Artificial Sequence
  Core Domain(R21N, S64K)
CPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 20 - protein, 106 aa, Artificial Sequence
  Core Domain(human GDF15 7-112aa)
CPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 21 - protein, 111 aa, Artificial Sequence
  GDF15 variant (delta N3, WS insertion, G4N, D5S, H6T/ FM1)
WSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 22 - protein, 109 aa, Artificial Sequence
  GDF15 variant (delta N3, G4N, D5S, H6T / FM2)
NSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 23 - protein, 110 aa, Artificial Sequence
  GDF15 variant (delta N2, R21N / FM3)
NGDHCPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 24 - protein, 110 aa, Artificial Sequence
  GDF15 variant (delta N2, N56L / FM4-3)
NGDHCPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 25 - protein, 110 aa, Artificial Sequence
  GDF15 variant (delta N2, S64K / FM5)
NGDHCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 26 - protein, 111 aa, Artificial Sequence
  GDF15 variant (delta N3, WS insertion, G4N, D5S, H6T, S64K / FM6)
WSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 27 - protein, 109 aa, Artificial Sequence
  GDF15 variant (delta N3, G4N, D5S, H6T, S64K / FM7)
NSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 28 - protein, 110 aa, Artificial Sequence
  GDF15 variant (delta N2, S64R / FM9)
NGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 29 - protein, 111 aa, Artificial Sequence
  GDF15 variant (delta N3, WS insertion, G4N, D5S, H6T, S64R/ FM10)
WSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 30 - protein, 109 aa, Artificial Sequence
  GDF15 variant (delta N3, G4N, D5S, H6T, S64R/ FM11)
NSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 31 - protein, 110 aa, Artificial Sequence
  GDF15 variant (delta N2, S64N/ FM13)
NGDHCPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 32 - protein, 110 aa, Artificial Sequence
  GDF15 variant (delta N2,S64D/ FM14)
NGDHCPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 33 - protein, 110 aa, Artificial Sequence
  GDF15 variant (delta N2,S64E/ FM15)
NGDHCPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 34 - protein, 110 aa, Artificial Sequence
  GDF15 variant (delta N2,S64L / FM16)
NGDHCPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 35 - protein, 110 aa, Artificial Sequence
  GDF15 variant (delta N2, D103L / FM4-2)
NGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 36 - protein, 110 aa, Artificial Sequence
  GDF15 variant (delta N2, N56C, D103C / FM4)
NGDHCPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 37 - protein, 110 aa, Artificial Sequence
  GDF15 variant (delta N2, N56S, D103S/ FM4-1)
NGDHCPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 38 - protein, 110 aa, Artificial Sequence
  GDF15 variant (delta N2, R21N, S64K / FM8)
NGDHCPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 39 - protein, 110 aa, Artificial Sequence
  GDF15 variant (delta N2, R21N, S64R / FM12)
NGDHCPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 40 - protein, 110 aa, Artificial Sequence
  GDF15 (delta N2)
NGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 41 - protein, 227 aa, Artificial Sequence
  Human IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 217 aa, Artificial Sequence
  Hinge deleted human IgG1-Fc(E233A, L235A) (T366W)-(A-1)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 217 aa, Artificial Sequence
  Hinge deleted human IgG1-Fc(E233A, L235A) (T366S, L368A, Y407V)-(A-2)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 217 aa, Artificial Sequence
  Hinge deleted human IgG1-Fc(E233A, L235A, N297A)(T366W)-(B-1)
APALAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 217 aa, Artificial Sequence
  Hinge deleted human IgG1-Fc(E233A, L235A, N297A)(T366S, L368A, Y407V)-(B-2)
APALAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 217 aa, Artificial Sequence
  Hinge deleted human IgG1-Fc(L234A, L235A, N297A)(T366W)-(C-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, Artificial Sequence
  Hinge deleted human IgG1-Fc(L234A, L235A, N297A)(T366S, L368A, Y407V)-(C-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 25 aa, Artificial Sequence
  linker-(G4S)5
GGGGSGGGGSGGGGSGGGGSGGGGS

SEQ ID NO: 49 - protein, 352 aa, Artificial Sequence
  Hinge deleted human IgG1-Fc(E233A, L235A)(T366W)-(G4S)5-GDF15(delta N2) - FWT
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 50 - protein, 353 aa, Artificial Sequence
  Hinge deleted human IgG1-Fc(E233A, L235A) (T366W)-(G4S)5-GDF15 variant (delta N3, WS insertion, G4N, D5S, H6T)-FM1
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 51 - protein, 351 aa, Artificial Sequence
  Hinge deleted human IgG1-Fc(E233A, L235A) (T366W)-(G4S)5-GDF15 variant (delta N3, G4N, D5S, H6T)-FM2
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 52 - protein, 352 aa, Artificial Sequence
  Hinge deleted human IgG1-Fc(E233A, L235A) (T366W)-(G4S)5-GDF15 variant (delta N2, R21N)-FM3
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 53 - protein, 352 aa, Artificial Sequence
  Hinge deleted human IgG1-Fc(E233A, L235A)(T366W)-(G4S)5-GDF15 variant (delta N2, N56L)-FM4-3
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 54 - protein, 352 aa, Artificial Sequence
  Hinge deleted human IgG1-Fc(E233A, L235A) (T366W)-(G4S)5-GDF15 variant (delta N2, S64K)-FM5
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 55 - protein, 353 aa, Artificial Sequence
  Hinge deleted human IgG1-Fc(E233A, L235A) (T366W)-(G4S)5-GDF15 variant (delta N3, WS insertion, G4N, D5S, H6T, S64K)-FM6
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 56 - protein, 351 aa, Artificial Sequence
  Hinge deleted human IgG1-Fc(E233A, L235A) (T366W)-(G4S)5-GDF15 variant (delta N3, G4N, D5S, H6T, S64K)-FM7
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 57 - protein, 352 aa, Artificial Sequence
  Hinge deleted human IgG1-Fc(E233A, L235A) (T366W)-(G4S)5-GDF15 variant (delta N2, S64R)-FM9
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 58 - protein, 353 aa, Artificial Sequence
  Hinge deleted human IgG1-Fc(E233A, L235A) (T366W)-(G4S)5-GDF15 variant (delta N3, WS insertion, G4N, D5S, H6T, S64R)-FM10
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 59 - protein, 351 aa, Artificial Sequence
  Hinge deleted human IgG1-Fc(E233A, L235A) (T366W)-(G4S)5-GDF15 variant (delta N3, G4N, D5S, H6T, S64R)-FM11
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 60 - protein, 352 aa, Artificial Sequence
  Hinge deleted human IgG1-Fc(E233A, L235A) (T366W)-(G4S)5-GDF15 variant (delta N2, S64N/ FM13)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 61 - protein, 352 aa, Artificial Sequence
  Hinge deleted human IgG1-Fc(E233A, L235A) (T366W)-(G4S)5-GDF15 variant (delta N2,S64D)-FM14
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 62 - protein, 352 aa, Artificial Sequence
  Hinge deleted human IgG1-Fc(E233A, L235A) (T366W)-(G4S)5-GDF15 variant (delta N2,S64E)-FM15
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 63 - protein, 352 aa, Artificial Sequence
  Hinge deleted human IgG1-Fc(E233A, L235A) (T366W)-(G4S)5-GDF15 variant (delta N2,S64L)-FM16
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 64 - protein, 352 aa, Artificial Sequence
  Hinge deleted human IgG1-Fc(E233A, L235A) (T366W)-(G4S)5-GDF15 variant (delta N2, D103L)-FM4-2
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 65 - protein, 352 aa, Artificial Sequence
  Hinge deleted human IgG1-Fc(E233A, L235A)(T366W)-(G4S)5-GDF15 variant (delta N2, N56C, D103C)-FM4
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 66 - protein, 352 aa, Artificial Sequence
  Hinge deleted human IgG1-Fc(E233A, L235A)(T366W)-(G4S)5-GDF15 variant (delta N2, N56S, D103S)-FM4-1
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 67 - protein, 352 aa, Artificial Sequence
  Hinge deleted human IgG1-Fc(E233A, L235A) (T366W)-(G4S)5-GDF15 variant (delta N2, R21N, S64K)-FM8
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 68 - protein, 352 aa, Artificial Sequence
  Hinge deleted human IgG1-Fc(E233A, L235A) (T366W)-(G4S)5-GDF15 variant (delta N2, R21N, S64R)-FM12
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 69 - protein, 352 aa, Artificial Sequence
  Hinge deleted human IgG1-Fc(E233A, L235A) (T366W)-(G4S)5-GDF15 variant (delta N2, W32F)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADFVLSPREVQVTMCIGACPSQFRAANMHAQIKTSLHRLKPDTVPAPCCVPASYNPMVLIQKTDTGVSLQTYDDLLAKDCHCI

SEQ ID NO: 70 - protein, 352 aa, Artificial Sequence
  Hinge deleted human IgG1-Fc(E233A, L235A) (T366W)-(G4S)5-GDF15 variant (delta N2, W32H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADHVLSPREVQVTMCIGACPSQFRAANMHAQIKTSLHRLKPDTVPAPCCVPASYNPMVLIQKTDTGVSLQTYDDLLAKDCHCI

SEQ ID NO: 71 - protein, 352 aa, Artificial Sequence
  Hinge deleted human IgG1-Fc(E233A, L235A) (T366W)-(G4S)5-GDF15 variant (delta N2, W32Y)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADYVLSPREVQVTMCIGACPSQFRAANMHAQIKTSLHRLKPDTVPAPCCVPASYNPMVLIQKTDTGVSLQTYDDLLAKDCHCI

SEQ ID NO: 72 - protein, 352 aa, Artificial Sequence
  Hinge deleted human IgG1-Fc(E233A, L235A) (T366W)-(G4S)5-GDF15 variant (delta N2, Q51H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSHFRAANMHAQIKTSLHRLKPDTVPAPCCVPASYNPMVLIQKTDTGVSLQTYDDLLAKDCHCI

SEQ ID NO: 73 - protein, 352 aa, Artificial Sequence
  Hinge deleted human IgG1-Fc(E233A, L235A) (T366W)-(G4S)5-GDF15 variant (delta N2, Q51L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSLFRAANMHAQIKTSLHRLKPDTVPAPCCVPASYNPMVLIQKTDTGVSLQTYDDLLAKDCHCI

SEQ ID NO: 74 - protein, 352 aa, Artificial Sequence
  Hinge deleted human IgG1-Fc(E233A, L235A) (T366W)-(G4S)5-GDF15 variant (delta N2, Q51E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSEFRAANMHAQIKTSLHRLKPDTVPAPCCVPASYNPMVLIQKTDTGVSLQTYDDLLAKDCHCI

SEQ ID NO: 75 - protein, 352 aa, Artificial Sequence
  Hinge deleted human IgG1-Fc(E233A, L235A) (T366W)-(G4S)5-GDF15 variant (delta N2, Q51N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSNFRAANMHAQIKTSLHRLKPDTVPAPCCVPASYNPMVLIQKTDTGVSLQTYDDLLAKDCHCI

SEQ ID NO: 76 - protein, 352 aa, Artificial Sequence
  Hinge deleted human IgG1-Fc(E233A, L235A) (T366W)-(G4S)5-GDF15 variant (delta N2, Q90H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIHKTDTGVSLQTYDDLLAKDCHCI

SEQ ID NO: 77 - protein, 352 aa, Artificial Sequence
  Hinge deleted human IgG1-Fc(E233A, L235A) (T366W)-(G4S)5-GDF15 variant (delta N2, Q90E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIEKTDTGVSLQTYDDLLAKDCHCI

SEQ ID NO: 78 - protein, 352 aa, Artificial Sequence
  Hinge deleted human IgG1-Fc(E233A, L235A) (T366W)-(G4S)5-GDF15 variant (delta N2, Q90K)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIKKTDTGVSLQTYDDLLAKDCHCI

SEQ ID NO: 79 - protein, 352 aa, Artificial Sequence
  Hinge deleted human IgG1-Fc(E233A, L235A) (T366W)-(G4S)5-GDF15 variant (delta N2, D93E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTETGVSLQTYDDLLAKDCHCI

SEQ ID NO: 80 - protein, 352 aa, Artificial Sequence
  Hinge deleted human IgG1-Fc(E233A, L235A) (T366W)-(G4S)5-GDF15 variant (delta N2, D93L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTLTGVSLQTYDDLLAKDCHCI

SEQ ID NO: 81 - protein, 352 aa, Artificial Sequence
  Hinge deleted human IgG1-Fc(E233A, L235A) (T366W)-(G4S)5-GDF15 variant (delta N2, D93N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTNTGVSLQTYDDLLAKDCHCI

SEQ ID NO: 82 - protein, 352 aa, Artificial Sequence
  Hinge deleted human IgG1-Fc(E233A, L235A) (T366W)-(G4S)5-GDF15 variant (delta N2, D93Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTQTGVSLQTYDDLLAKDCHCI

SEQ ID NO: 83 - protein, 352 aa, Artificial Sequence
  Hinge deleted human IgG1-Fc(E233A, L235A) (T366W)-(G4S)5-GDF15 variant (delta N2, Q60L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHALIKTSLHRLKPDTVPAPCCVPASYNPMVLIQKTDTGVSLQTYDDLLAKDCHCI

SEQ ID NO: 84 - protein, 352 aa, Artificial Sequence
  Hinge deleted human IgG1-Fc(E233A, L235A) (T366W)-(G4S)5-GDF15 variant (delta N2,Q60N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHANIKTSLHRLKPDTVPAPCCVPASYNPMVLIQKTDTGVSLQTYDDLLAKDCHCI

SEQ ID NO: 85 - protein, 352 aa, Artificial Sequence
  Hinge deleted human IgG1-Fc(E233A, L235A) (T366W)-(G4S)5-GDF15 variant (delta N2, S64Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTQLHRLKPDTVPAPCCVPASYNPMVLIQKTDTGVSLQTYDDLLAKDCHCI

SEQ ID NO: 86 - protein, 352 aa, Artificial Sequence
  Hinge deleted human IgG1-Fc(E233A, L235A) (T366W)-(G4S)5-GDF15 variant (delta N2, T92S)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKSDTGVSLQTYDDLLAKDCHCI

SEQ ID NO: 87 - protein, 352 aa, Artificial Sequence
  Hinge deleted human IgG1-Fc(E233A, L235A) (T366W)-(G4S)5-GDF15 variant (delta N2, T92E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKEDTGVSLQTYDDLLAKDCHCI

SEQ ID NO: 88 - protein, 352 aa, Artificial Sequence
  Hinge deleted human IgG1-Fc(E233A, L235A) (T366W)-(G4S)5-GDF15 variant (delta N2, S97N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVNLQTYDDLLAKDCHCI

SEQ ID NO: 89 - protein, 352 aa, Artificial Sequence
  Hinge deleted human IgG1-Fc(E233A, L235A) (T366W)-(G4S)5-GDF15 variant (delta N2, S97Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVQLQTYDDLLAKDCHCI

SEQ ID NO: 90 - protein, 352 aa, Artificial Sequence
  Hinge deleted human IgG1-Fc(E233A, L235A) (T366W)-(G4S)5-GDF15 variant (delta N2,Y101F)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTFDDLLAKDCHCI

SEQ ID NO: 91 - protein, 352 aa, Artificial Sequence
  Hinge deleted human IgG1-Fc(E233A, L235A) (T366W)-(G4S)5-GDF15 variant (delta N2, Y101Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTQDDLLAKDCHCI

SEQ ID NO: 92 - protein, 17 aa, Artificial Sequence
  linker-GS(G4S)3
GSGGGGSGGGGSGGGGS

SEQ ID NO: 93 - protein, 27 aa, Artificial Sequence
  linker-GS(G4S)5
GSGGGGSGGGGSGGGGSGGGGSGGGGS

SEQ ID NO: 94 - protein, 37 aa, Artificial Sequence
  linker-GS(G4S)7
GSGGGGSGGGGSGGGGSGGGGSGGGGSGGGGSGGGGS

SEQ ID NO: 95 - protein, 26 aa, Artificial Sequence
  linker-GS(EEEA)6
GSEEEAEEEAEEEAEEEAEEEAEEEA

SEQ ID NO: 96 - protein, 26 aa, Artificial Sequence
  linker-GSGGSS(PT)10
GSGGSSPTPTPTPTPTPTPTPTPTPT

SEQ ID NO: 97 - protein, 27 aa, Artificial Sequence
  linker-GS(EAAAK)5
GSEAAAKEAAAKEAAAKEAAAKEAAAK

SEQ ID NO: 98 - protein, 344 aa, Artificial Sequence
  Hinge deleted human IgG1-Fc(L234A, L235A, N297A)(T366W)-GS(G4S)3-GDF15 variant (delta N2, S64R / FM9-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 99 - protein, 354 aa, Artificial Sequence
  Hinge deleted human IgG1-Fc(L234A, L235A, N297A)(T366W)-GS(G4S)5-GDF15 variant (delta N2, S64R / FM9-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 100 - protein, 364 aa, Artificial Sequence
  Hinge deleted human IgG1-Fc(L234A, L235A, N297A)(T366W)-GS(G4S)7-GDF15 variant (delta N2, S64R / FM9-3)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 101 - protein, 353 aa, Artificial Sequence
  Hinge deleted human IgG1-Fc(L234A, L235A, N297A)(T366W)-GS(EEEA)6-GDF15 variant (delta N2, S64R / FM9-4)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEEEAEEEAEEEAEEEAEEEAEEEANGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 102 - protein, 353 aa, Artificial Sequence
  Hinge deleted human IgG1-Fc(L234A, L235A, N297A)(T366W)-GSGGSS(PT)10-GDF15 variant (delta N2, S64R / FM9-5)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGSSPTPTPTPTPTPTPTPTPTPTNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 103 - protein, 354 aa, Artificial Sequence
  Hinge deleted human IgG1-Fc(L234A, L235A, N297A)(T366W)-GS(EAAAK)5-GDF15 variant (delta N2, S64R / FM9-6)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEAAAKEAAAKEAAAKEAAAKEAAAKNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 104 - protein, 343 aa, Artificial Sequence
  Hinge deleted human IgG1-Fc(L234A, L235A, N297A)(T366W)-GS(G4S)3-GDF15 variant (delta N3, G4N, D5S, H6T, S64R/ FM11-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 105 - protein, 353 aa, Artificial Sequence
  Hinge deleted human IgG1-Fc(L234A, L235A, N297A)(T366W)-GS(G4S)5-GDF15 variant (delta N3, G4N, D5S, H6T, S64R/ FM11-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 106 - protein, 363 aa, Artificial Sequence
  Hinge deleted human IgG1-Fc(L234A, L235A, N297A)(T366W)-GS(G4S)7-GDF15 variant (delta N3, G4N, D5S, H6T, S64R/ FM11-3)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 107 - protein, 352 aa, Artificial Sequence
  Hinge deleted human IgG1-Fc(L234A, L235A, N297A)(T366W)-GS(EEEA)6-GDF15 variant (delta N3, G4N, D5S, H6T, S64R/ FM11-4)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEEEAEEEAEEEAEEEAEEEAEEEANSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 108 - protein, 352 aa, Artificial Sequence
  Hinge deleted human IgG1-Fc(L234A, L235A, N297A)(T366W)-GSGGSS(PT)10-GDF15 variant (delta N3, G4N, D5S, H6T, S64R/ FM11-5)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGSSPTPTPTPTPTPTPTPTPTPTNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 109 - protein, 353 aa, Artificial Sequence
  Hinge deleted human IgG1-Fc(L234A, L235A, N297A)(T366W)-GS(EAAAK)5-GDF15 variant (delta N3, G4N, D5S, H6T, S64R/ FM11-6)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEAAAKEAAAKEAAAKEAAAKEAAAKNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 110 - protein, 39 aa, Artificial Sequence
  Exenatide
HGEGTFTSDLSKQMEEEAVRLFIEWLKNGGPSSGAPPPS

SEQ ID NO: 111 - protein, 32 aa, Artificial Sequence
  Liraglutide
HAEGTFTSDVSSYLEGQAAKEEFIAWLVRGRG

SEQ ID NO: 112 - protein, 44 aa, Artificial Sequence
  lixisenatide
HGEGTFTSDLSKQMEEEAVRLFIEWLKNGGPSSGAPPSKKKKKK

SEQ ID NO: 113 - protein, 30 aa, Artificial Sequence
  taspoglutide
  misc_feature (2)..(2): Xaa can be any naturally occurring amino acid
  misc_feature (29)..(29): Xaa can be any naturally occurring amino acid
HXEGTFTSDVSSYLEGQAAKEFIAWLVKXR

SEQ ID NO: 114 - protein, 31 aa, Artificial Sequence
  Semaglutide
  misc_feature (2)..(2): Xaa can be any naturally occurring amino acid
HXEGTFTSDVSSYLEGQAAKEFIAWLVRGRG

SEQ ID NO: 115 - protein, 275 aa, Artificial Sequence
  Dulaglutide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 645 aa, Artificial Sequence
  Albiglutide
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 117 - protein, 39 aa, Artificial Sequence
  Tirzepatide
  misc_feature (2)..(2): Xaa can be any naturally occurring amino acid
  misc_feature (13)..(13): Xaa can be any naturally occurring amino acid
YXEGTFTSDYSIXLDKIAQKAFVQWLIAGGPSSGAPPPS

SEQ ID NO: 118 - protein, 30 aa, Artificial Sequence
  Cotadutide
HSQGTFTSDKSEYLDSERARDFVAWLEAGG